(19) United States
(12) Patent Application Publication
(10) Pub. No.: US 2025/0116636 A1
(43) Pub. Date: Apr. 10, 2025

(54) METHODS AND DEVICES FOR METABOLOMICS AND LIPIDOMICS ANALYSIS

(71) Applicant: Seer, Inc., Redwood City, CA (US)

(72) Inventors: Daniel HORNBURG, Foster City, CA (US); Mathew ELLENBERGER, San Francisco, CA (US); Brittany LEE, San Carlos, CA (US)

(21) Appl. No.: 18/730,925

(22) PCT Filed: Feb. 2, 2023
(86) PCT No.: PCT/US2023/061869

§ 371 Date: Jul. 22, 2024

Related U.S. Application Data
(60) Provisional application No. 63/306,469, filed on Feb. 3, 2022.
(60) Provisional application No. 63/479,869, filed on Jan. 13, 2023.

(51) Int. Cl. G01N 30/06; G01N 30/88; G01N 33/68; G01N 33/92

(52) U.S. Cl. CPC G01N 30/06; G01N 33/6848; G01N 33/92; G01N 2030/065; G01N 2030/8831; G01N 2405/04

(57) ABSTRACT

Described herein are methods of assaying a biological sample, including adding a solvent to a composition of the biological sample to generate a mixture of an organic layer and an aqueous layer. The organic layer contains at least one lipid. The aqueous layer contains at least one metabolite. Also, described herein are methods of assaying a biological sample, including performing mass spectrometry on at least a portion of an organic layer to identify at least one lipid. Also, described herein are methods of assaying a biological sample, including performing mass spectrometry on at least a portion of an aqueous layer to identify at least one metabolite. Also, described herein are apparatus and kits for assaying a biological sample including at least one protein and at least one lipid or metabolite.

### CROSS-REFERENCE

This application claims the benefit of U.S. Provisional Patent Application No. 63/306,469, filed Feb. 3, 2022, and U.S. Provisional Patent Application No. 63/479,869, filed Jan. 13, 2023, each of which applications is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Lipids and metabolites are each structurally and functionally diverse classes of biological molecules. As a result, isolation and identification of lipids and/or metabolites from biological samples is challenging.

### SUMMARY

An aspect of the present disclosure provides a method of assaying a biological sample, comprising: (a) providing the biological sample comprising a plurality of biomolecules, wherein the plurality of biomolecules comprises at least one protein and at least one lipid or metabolite; (b) contacting the biological sample with a surface to bind the at least one protein, thereby yielding an altered composition of the biological sample, wherein the altered composition of the biological sample comprises a reduced amount of the at least one protein; (c) adding a first solvent to the altered composition of the biological sample to generate a mixture comprising a first organic layer and an aqueous layer, wherein the at least one lipid or metabolite partitions into the first organic layer; and (d) performing mass spectrometry on at least a portion of the first organic layer, thereby identifying the at least one lipid or metabolite. In some embodiments, (b) further comprises separating the surface and the altered composition of the biological sample.

Another aspect of the present disclosure provides a method of assaying a biological sample, comprising: (a) providing the biological sample comprising a plurality of biomolecules, wherein the plurality of biomolecules comprises at least one protein and at least one lipid or metabolite; (b) adding a first solvent to the biological sample to generate a mixture comprising a first organic layer and an aqueous layer, wherein the at least one lipid or metabolite partitions into the first organic layer; (c) contacting at least a portion of the first organic layer or the aqueous layer with a surface, wherein the at least one protein binds to the surface; and (d) performing mass spectrometry on at least a portion of the first organic layer, thereby identifying the at least one lipid or metabolite. In some embodiments, the method further comprises after the contacting, separating the first organic layer or the aqueous layer from the surface.

In some aspects, the present disclosure provides a method of assaying a biological sample, comprising: (a) providing the biological sample comprising a plurality of biomolecules, wherein the plurality of biomolecules comprises at least one lipid or metabolite; (b) contacting at least a portion of the sample with a surface to adsorb the at least one lipid or metabolite on the surface; (c) separating the at least one lipid or metabolite from the surface to generate an isolated lipid or metabolite; and (d) performing mass spectrometry on the isolated lipid or metabolite, thereby identifying the at least one lipid or metabolite.

In some embodiments, the separating comprises contacting the at least one lipid or metabolite with a first solvent. In some embodiments, the biological sample further comprises at least one protein. In some embodiments, the method further comprises performing mass spectrometry on the at least one protein. In some embodiments, the method further comprises contacting at least another portion of the sample with a second surface, wherein the second surface is configured to bind the at least one protein. In some embodiments, the contacting the at least another portion of the sample with a second surface is performed prior to the contacting in (b). In some embodiments, the contacting the at least another portion of the sample with the second surface is performed subsequent to the contacting in (b). In some embodiments, a density of the first solvent is lower than a density of water. In some embodiments, a density of the first solvent is higher than a density of water. In some embodiments, a dielectric constant of the first solvent is lower than a dielectric constant of water. In some embodiments, the dielectric constant of the first solvent is higher than the dielectric constant of water. In some embodiments, a dielectric constant of the first solvent is less than about 1, about 2, about 3, about 4, about 5, about 6, about 7, about 8, about 9, about 10, about 20, about 30, about 40, about 50, about 60, about 70, or about 80 at about 20° C. In some embodiments, a viscosity of the first solvent is greater than about 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, or 0.8 mPa·s. In some embodiments, a vapor pressure of the first solvent is less than about 400, about 300, about 250, about 200, about 100, about 90, about 80, about 70, about 60, or about 50 torr at about 20° C. In some embodiments, the first solvent comprises an alcohol, an aliphatic hydrocarbon, an aromatic hydrocarbon, an aliphatic heterocyclic compound, an aromatic heterocyclic compound, an amide, an ester, an ether, a ketone, a halocarbon, or a nitrile, or any combination thereof. In some embodiments, the first solvent comprises an ester or ether. In some embodiments, the first solvent comprises ethyl acetate, propyl acetate, butyl acetate, or amyl acetate, or any isomer or combination thereof. In some embodiments, the first solvent comprises butyl acetate. In some embodiments, the first solvent comprises diethyl ether, methyl tert-butyl ether (MTBE), dioxane, tetrahydrofuran (THF), or anisole, or any isomer or combination thereof. In some embodiments, the first solvent comprises MTBE. In some embodiments, the first solvent does not comprise a halocarbon. In some embodiments, the first solvent comprises an alcohol. In some embodiments, the first solvent comprises methanol, ethanol, propanol, butanol, pentanol, or hexanol, or any isomer or combination thereof. In some embodiments, the first solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some embodiments, the first solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some embodiments, the second solvent substantially partitions into the first organic layer. In some embodiments, the second solvent substantially partitions into a second organic layer. In some embodiments, the first solvent comprises an alcohol and the second solvent comprises an ester. In some embodiments, the ester comprises butyl acetate. In some embodiments, the alcohol comprises butanol, methanol, or a combination thereof. In some embodiments, the first solvent comprises an alcohol and the second solvent comprises an ester and an ether. In some embodiments, the ether comprises methyl tert-butyl ether (MTBE). In some embodiments, the ester comprises butyl acetate. In some embodiments, the method further comprises adding a pH adjusting agent to the altered composition of the biological sample. In some embodiments, the pH adjusting agent comprises a base. In some embodiments, the pH adjusting agent comprises an acid. In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample comprises blood, serum, or plasma, or any portion of fraction thereof. In some embodiments, the biological sample comprises plasma. In some embodiments, the plasma is diluted. In some embodiments, the method further comprises purifying the at least one lipid or metabolite from the first organic layer. In some embodiments, the at least one lipid comprises a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a sphingomyelin (SM), a cardiolipin (CL), or a free fatty acid (FFA), or any derivative thereof. In some embodiments, the at least one lipid comprises a ChE, a CER, a CL, a DAG, a LPC, a LPE, a PG, a PE, a PI, a SM, or a TAG. In some embodiments, the at least one lipid is comprised in a plurality of lipids in the biological sample. In some embodiments, the plurality of lipids comprises a dynamic range of at least 1, 2, 3, 4, 5, 6, 7, 8, 9, 10, 11, 12, 13, or 14 orders of magnitude in the biological sample. In some embodiments, the plurality of lipids comprises a dynamic range of no more than about 14, 13, 12, 11, 10, 9, 8, 7, 6, 5, 4, 3, 2, or 1 orders of magnitude in the biological sample. In some embodiments, at least a portion of the plurality of lipid comprises a dynamic range in the first organic layer which is less than a dynamic range of the plurality of lipids in the biological sample. In some embodiments, the at least one metabolite comprises a vitamin, a cofactor, a nucleotide, a polynucleotide, an amino acid or analogue thereof, a peptidomimetic, an organic acid, an alcohol, a diol, a polyol, a monosaccharide, a disaccharide, an oligosaccharide, a polysaccharide, a hydrocarbon, a benzenoid, an alkaloid, an acetylide, a polyketide, a terpene or terpenoid, a phenolic, or any combination or derivative thereof. In some embodiments, the first surface is a surface of a particle. In some embodiments, the particle is a nanoparticle. In some embodiments, the particle is a microparticle. In some embodiments, the particle is a micelle, liposome, iron oxide particle, silver particle, gold particle, palladium particle, quantum dot, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, 2-(3-aminopropylamino) ethanol particle, protein functionalized particle, ubiquitin functionalized particle, polysaccharide coated particle, or dextran functionalized particle. In some embodiments, the particle is a magnetic particle. In some embodiments, the magnetic particle is a superparamagnetic iron oxide particle. In some embodiments, the particle comprises an iron oxide material. In some embodiments, the particle comprises an iron oxide core. In some embodiments, the particle comprises iron oxide crystals embedded in a polystyrene core. In some embodiments, the particle comprises a polymer coating. In some embodiments, the particle comprises a positively charged polymer, a negatively charged polymer, a zwitterionic polymer, or any combination thereof. In some embodiments, the particle a silica shell coating. In some embodiments, the particle comprises a poly(N-(3-(dimethylamino)propyl) methacrylamide) (PDMAPMA) coating. In some embodiments, the particle comprises a poly(oligo(ethylene glycol) methyl ether methacrylate) (POEGMA) coating. In some embodiments, the particle comprises a positive surface charge. In some embodiments, the particle comprises a negative surface charge. In some embodiments, the particle comprises a neutral surface charge. In some embodiments, the contacting comprises contacting the sample, the first organic layer, or the aqueous layer with a plurality of surface regions. In some embodiments, the plurality of surface regions is disposed on the first surface. In some embodiments, the plurality of surface regions are disposed on a plurality of discrete surfaces comprising the first surface. In some embodiments, a first surface region of the plurality of surface regions comprises a first physiochemical property and a second surface region of the plurality of surface regions comprises a second physicochemical property different from the first physicochemical property. In some embodiments, the first physicochemical property comprises charge, zeta potential, hydrophobicity, surface functional group, or any combination thereof. In some embodiments, the plurality of discrete surfaces are surfaces of a plurality of particles. In some embodiments, the first physicochemical property comprises size, charge, core material, shell material, porosity, zeta potential, hydrophobicity, surface functional group, or any combination thereof. In some embodiments, the plurality of surface regions is comprised on an array. In some embodiments, the biological sample comprises at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids or metabolites. In some embodiments, the biological sample comprises no more than about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites. In some embodiments, the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids or metabolites. In some embodiments, the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids. In some embodiments, the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct metabolites. In some embodiments, the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites. In some embodiments, the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids. In some embodiments, the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct metabolites. In some embodiments, the performing mass spectrometry comprises identifying at least about 10, 20, 30, 40, 50, 60, 70, 80, 90, 100, or more cholesteryl esters (ChEs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more ceramides (CERs) in the biological sample. In some embodiments, the performing mass spectrometry comprises identifying at least about 10, 20, 30, 40, 50, 60, 70, 80, 90, 100, or more cardiolipins (CLs) in the biological sample. In some embodiments, the performing mass spectrometry comprises identifying at least about 10, 20, 30, 40, 50, 60, 70, 80, 90, 100, or more diacyl glycerols (DAGs) in the biological sample. In some embodiments, the performing mass spectrometry comprises identifying at least about 10, 20, 30, 40, 50, 60, 70, 80, 90, 100, or more lysophosphatidylcholines (LPCs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more phosphatidylcholines (PCs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more phosphatidylethanolamines (PEs) in the biological sample. In some embodiments, the performing mass spectrometry comprises identifying at least about 10, 20, 30, 40, 50, 60, 70, 80, 90, 100, or more phosphatidylinositols (PIS) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more sphingomyelins (SMs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more triacylglycerols (TAGs) in the biological sample.

In some aspects, the present disclosure provides a method of assaying a biological sample, comprising: (a) providing the biological sample comprising a plurality of biomolecules, wherein the plurality of biomolecules comprises at least one lipid; (b) adding a first solvent to the biological sample to generate a mixture comprising a first organic layer and an aqueous layer, wherein the first organic layer comprises the at least one lipid, and wherein the first solvent comprises butyl acetate; and (c) performing mass spectrometry on the first organic layer, thereby identifying the at least one lipid.

In some embodiments, the biological sample further comprises at least one protein. In some embodiments, the method further comprises, contacting the biological sample, the first organic layer, or the aqueous layer with a surface, wherein the at least one protein binds to the surface. In some embodiments, the method further comprises performing mass spectrometry on the at least one protein. In some embodiments, the first surface is a surface of particle. In some embodiments, the particle is a nanoparticle. In some embodiments, the particle is a microparticle. In some embodiments, the contacting comprises contacting the sample, the first organic layer, or the aqueous layer with a plurality of surface regions. In some embodiments, the plurality of surface regions is disposed on the surface. In some embodiments, the plurality of surface regions are disposed on a plurality of discrete surfaces comprising the surface. In some embodiments, first surface region of the plurality of surface regions comprises a first physiochemical property and a second surface region of the plurality of surface regions comprises a second physicochemical property different from the first physicochemical property. In some embodiments, the first physicochemical property comprises charge, zeta potential, hydrophobicity, surface functional group, or any combination thereof. In some embodiments, the plurality of discrete surfaces are surfaces of a plurality of particles. In some embodiments, the wherein the first physicochemical property comprises size, charge, core material, shell material, porosity, zeta potential, hydrophobicity, surface functional group, or any combination thereof. In some embodiments, the plurality of surface regions is comprised on an array. In some embodiments, the method further comprises adding water to the biological sample. In some embodiments, the first solvent further comprises an alcohol, an aliphatic hydrocarbon, an aromatic hydrocarbon, an aliphatic heterocyclic compound, an aromatic heterocyclic compound, an amide, an ester, an ether, a ketone, a halocarbon, or a nitrile, or any combination thereof. In some embodiments, the first solvent comprises an ester. In some embodiments, the first solvent comprises diethyl ether, methyl tert-butyl ether (MTBE), dioxane, tetrahydrofuran (THF), or anisole, or any isomer or combination thereof. In some embodiments, the first solvent comprises MTBE. In some embodiments, the first solvent does not comprise a halocarbon. In some embodiments, the first solvent comprises an alcohol. In some embodiments, the first solvent comprises methanol, ethanol, propanol, butanol, pentanol, or hexanol, or any isomer or combination thereof. In some embodiments, the first solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some embodiments, the first solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some embodiments, the method further comprises adding a second solvent to the biological sample, the first organic layer or the aqueous layer. In some embodiments, the second solvent substantially partitions into the first organic layer. In some embodiments, the second solvent substantially partitions into a second organic layer. In some embodiments, the second solvent comprises an alcohol. In some embodiments, the second solvent comprises methanol, ethanol, propanol, butanol, pentanol, or hexanol, or any isomer or combination thereof. In some embodiments, the second solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some embodiments, the second solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some embodiments, the second solvent comprises an ester. In some embodiments, the second solvent comprises methyl tert-butyl ether (MTBE). In some embodiments, the method further comprises adding a pH adjusting agent to the biological sample, the first organic layer, the second organic layer, or the aqueous layer. In some embodiments, the pH adjusting agent comprises a base. In some embodiments, the pH adjusting agent comprises an acid. In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample comprises blood, serum, or plasma, or any portion of fraction thereof. In some embodiments, the biological sample comprises plasma. In some embodiments, the method further comprises purifying the at least one lipid from the first organic layer. In some embodiments, the at least one lipid comprises a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a cardiolipin (CL), or a sphingomyelin (SM), or any derivative thereof. In some embodiments, the at least one lipid comprises a ChE, a CER, a CL, a DAG, a LPC, a LPE, a PG, a PE, a PI, a SM, or a TAG. In some embodiments, the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids. In some embodiments, the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids. In some embodiments, the performing mass spectrometry comprises identifying at least about 10, 20, 30, 40, 50, 60, 70, 80, 90, 100, or more cholesteryl esters (ChEs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more ceramides (CERs) in the biological sample. In some embodiments, the performing mass spectrometry comprises identifying at least about 10, 20, 30, 40, 50, 60, 70, 80, 90, 100, or more cardiolipins (CLs) in the biological sample. In some embodiments, the performing mass spectrometry comprises identifying at least about 10, 20, 30, 40, 50, 60, 70, 80, 90, 100, or more diacyl glycerols (DAGs) in the biological sample. In some embodiments, the performing mass spectrometry comprises identifying at least about 10, 20, 30, 40, 50, 60, 70, 80, 90, 100, or more lysophosphatidylcholines (LPCs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more phosphatidylcholines (PCs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more phosphatidylethanolamines (PEs) in the biological sample. In some embodiments, the performing mass spectrometry comprises identifying at least about 10, 20, 30, 40, 50, 60, 70, 80, 90, 100, or more phosphatidylinositols (PIs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, or more sphingomyelins (SMs) in the biological sample. In some embodiments, the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more triacylglycerols (TAGs) in the biological sample.

In some aspects, the present disclosure provides an apparatus for assaying a biological sample comprising at least one protein and at least one lipid or metabolite, the apparatus comprising: a substrate comprising a surface; a loading unit that is operably coupled to the substrate; and a computer-readable medium comprising machine-executable code that, upon execution by one or more processors programmed individually or collectively, implements a method comprising: transferring at least a portion of the biological sample to the surface, thereby contacting the at least the portion of the biological sample with the surface to bind the at least one protein and yield an altered composition of the at least the portion of the biological sample, wherein the altered composition of the biological sample comprises a reduced amount of the at least one protein; and adding a first solvent to at least a portion of the altered composition of the biological sample to generate a mixture comprising a first organic layer and an aqueous layer, wherein the at least one lipid or metabolite partitions into the first organic layer.

In some embodiments, the method further comprises transferring at least a portion of the mixture to a container. In some embodiments, the at least a portion of the mixture comprises at least a portion of the organic layer. In some embodiments, the at least a portion of the mixture comprises at least a portion of the aqueous layer. In some embodiments, the first solvent comprise butyl acetate.

In some aspects, the present disclosure provides a kit for assaying a biological sample, comprising: a substrate comprising a surface, wherein the substrate comprises a surface configured to bind a biomolecule in the biological sample; and a first organic solvent.

In some embodiments, the first organic solvent comprises butyl acetate.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative cases, in which the principles of the disclosure are utilized, and the accompanying drawings of which:

FIGS. 1A-1C illustrate representative workflows for assaying biological samples, in accordance with some embodiments of the disclosure.

FIG. 1D illustrates an extraction of lipids and metabolites using an organic solvent and water, in accordance with some embodiments of the disclosure.

FIG. 2 illustrates an example of sample preparation steps in accordance with some embodiments of the disclosure.

FIG. 3 illustrates an example of a sample preparation workflow in accordance with some embodiments of the disclosure.

FIG. 4 illustrates an example sample analysis workflow in accordance with some embodiments of the disclosure.

FIG. 5 illustrates a graph showing counts of lipid species by class identified by a method as disclosed herein.

FIGS. 6A-6C show additional graphs of lipids by class identified adsorbed to nanoparticles as described herein.

FIGS. 7A and 7B illustrate box plots showing relative amounts of lipids across various classes identified adsorbed to nanoparticles as disclosed herein.

FIG. 8 depicts Venn diagrams showing lipids denied by class on nanoparticles as disclosed herein.

FIG. 9 depicts a volcano pot showing metabolites extracted by different workflows in accordance with the present disclosure.

FIG. 10 depicts a scores plots of samples processed in accordance with different metabolomics workflows of the present disclosure.

FIG. 11 depicts distributions of coefficients of variation (CVs) as calculated for several replicates of metabolomics workflows in accordance with the disclosure.

FIG. 12A depicts a plot of mean intensities (left) and a distribution of CVs (right) for lipids detected across two different lipidomics workflows of the present disclosure.

FIG. 12B depicts bar plots (left and right) and a Venn diagram (center) illustrating different lipid identified across two different lipidomics workflows as described herein.

FIG. 13 illustrates the number of lipid species by lipid class identified by various lipidomics workflows as described herein.

FIGS. 14A and 14B illustrate lipidomics PCAs of Comparison 3 (1335 MSMS Annotated lipids, class >15 lipids) (FIG. 14A) and Sample 1 (Methods in positive methods; 910 MSMS Annotated lipids) (FIG. 14B), comparing different buffer components, including BUME butyl, BUME heptane, BUME Single Phase, ethanol, and MTBE.

FIGS. 15A and 15B illustrate lipidomics CVs (%) of Comparison 3 (FIG. 15A) and Sample 1 (FIG. 15B), comparing different buffer components, including BUME butyl, BUME heptane, BUME Single Phase, ethanol, and MTBE.

FIGS. 16A-16C depict bar graphs showing different numbers and classes of metabolites identified across different metabolomics workflows as described herein. FIG. 16A illustrates the difference according to preparation type. FIGS. 16B and 16C illustrate the differences according to chromatography mode.

FIGS. 17A and 17B illustrates metabolomics PCAs of Comparison 3 (n=356) (FIG. 17A) and Sample 1 (n=990) (FIG. 17B), comparing different buffer components, including BUME butyl, BUME heptane, methanol, and MTBE.

FIGS. 18A and 18B illustrates metabolomics CVs (%) of Comparison 3 (FIG. 18A) and Sample 1 (FIG. 18B), comparing different buffer components, including BUME butyl, BUME heptane, methanol, and MTBE.

FIGS. 19A and 19B illustrate metabolomics PCAs of Sample 1, comparing different buffer components, including BUME butyl, BUME heptane, methanol, and MTBE, in HILIC (n=320) (FIG. 19A) and RPLC (n=250) (FIG. 19B) chromatography modes.

FIG. 20 shows a computer system that is programmed or otherwise configured to implement methods provided herein.

### DETAILED DESCRIPTION

### Overview

Metabolomics is the study of chemical processes involving metabolites, the small molecule substrates, intermediates, and products of cell metabolism. The “metabolome” as used herein generally refers to the complete set of metabolites (e.g., metabolic intermediates, hormones and other signaling molecules, and secondary metabolites) in a biological cell, tissue, organ, or organism.

Lipidomics is the large-scale study of pathways and networks of cellular lipids in biological systems. The term “lipidome” as used herein generally refers to the complete lipid profile within a cell, tissue, organism, or ecosystem. In some cases, a lipidome is a subset of the metabolome. Lipidomics generally involves the identification and quantification of cellular lipid molecular species and their interactions with other lipids, proteins, and other metabolites. Compared to other classes of biological macromolecules (e.g., proteins, nucleic acids), lipids and metabolites are much more structurally heterogenous and correspondingly display a wider variation in physiochemical properties. This structural, functional, and chemical heterogeneity can present challenges for workflows looking to identify and quantify a wide breadth of lipids and/or metabolites in a biological sample.

Representative lipidomic or metabolic analyses can comprise extraction with an appropriate solvent system before detection and analysis (e.g., by mass spectrometry). The solvent system may extract lipids without inducing or promoting the degradation of lipids or introducing contamination by non-lipid components. Thus, identification and profiling of lipids may depend on the efficiency of the extraction step. Poor extraction may cause loss of sensitivity, reproducibility, accuracy, and precision of the detection and analysis of any lipid in a sample. Further, the extraction solvent system may be able to solvate lipids across the full range of chemically diverse lipids. Also, the extraction procedure should be able to eliminate particulate matter, reduce chemical and matrix effects, and deliver the target compounds in solution at certain concentrations suitable for subsequent detection and analysis.

Documented methods and compositions for lipid extraction and isolation from biological samples exploit the high solubility of hydrocarbon chains in organic solvents, such as dichloromethane (DCM), methyl tert-butyl ether (MTBE), or chloroform. However, these methods and compositions are limited in their ability to be automated because of their toxicity, handling problems (e.g., drips from pipette), incompatibility with plastic materials, etc. To overcome one or more of these drawbacks, disclosed herein are methods of assaying a biological sample that may be comprised in or are compatible with automated multiomics workflows. Also, disclosed herein are methods and systems for extracting or separating lipids, metabolites, or proteins for manual and automated analytic (e.g., multiomics) workflows capturing biomolecules exhibiting a broad range of physicochemical properties.

### Systems and Methods for Analyzing Biological Samples

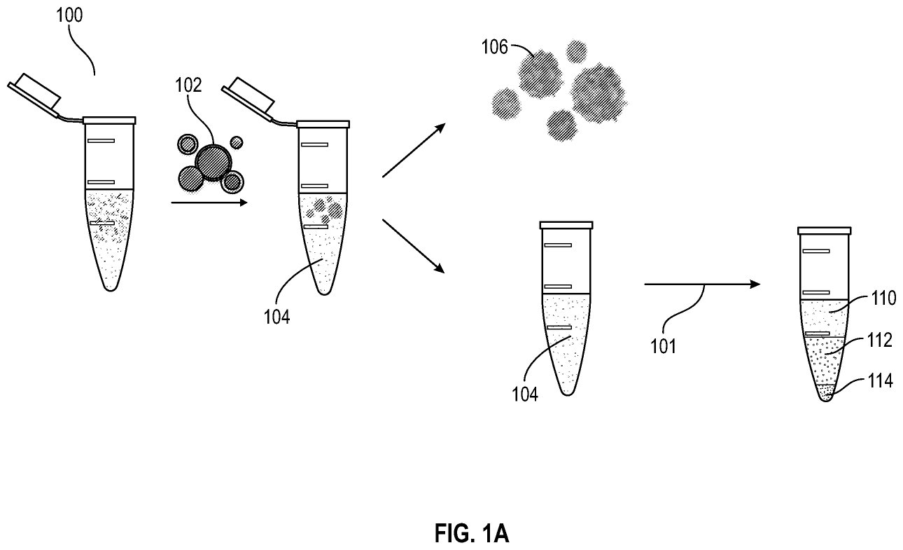

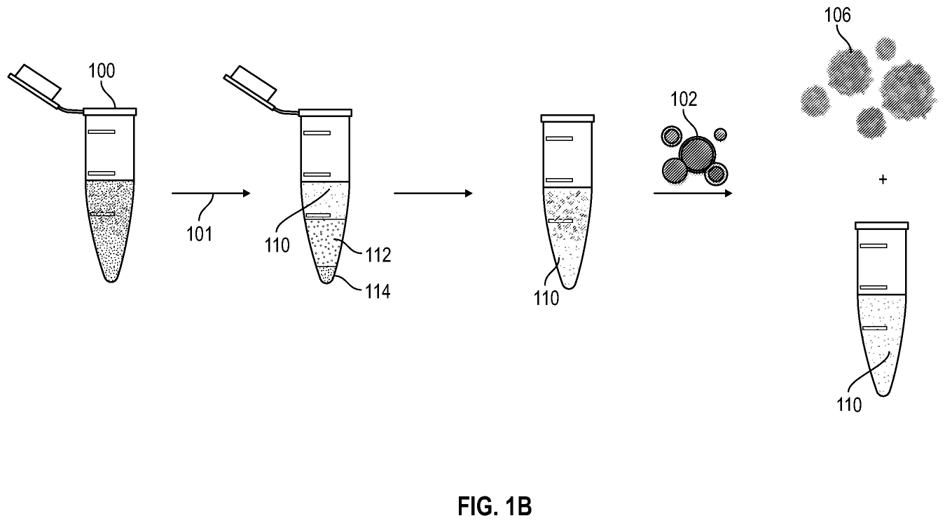

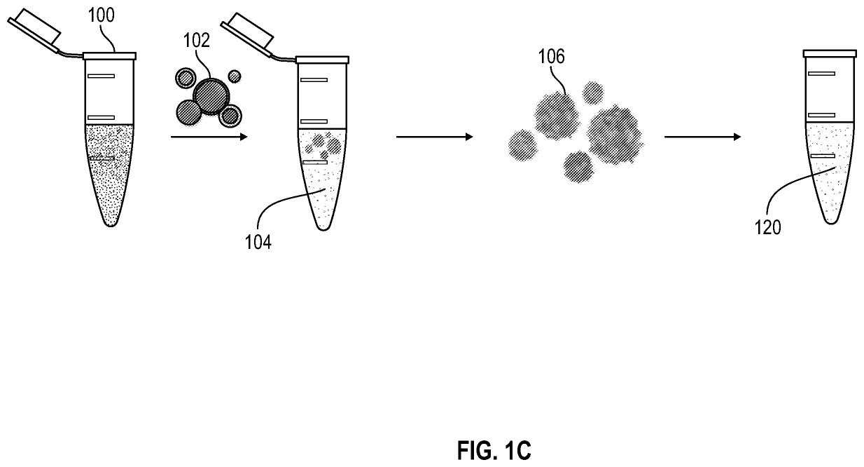

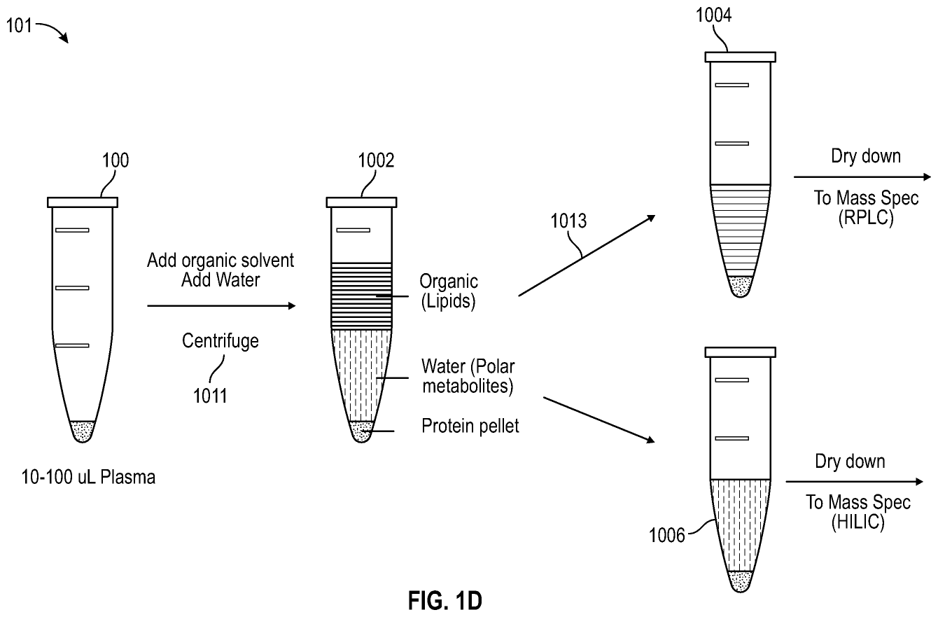

In some aspects, provided herein are methods of assaying a biological sample. FIGS. 1A-1C illustrate parts of representative workflows for assaying a biological sample, in accordance with some cases of the present disclosure.

The methods may comprise an operation of providing the biological sample. The biological sample may comprise a plurality of biomolecules, including proteins, nucleic acid, lipids, metabolites, and fragments or derivatives thereof. In some cases, one or more of these classes of biomolecules (or a subset thereof), may be separated from the others in order to facilitate downstream analysis (e.g., identification or quantification).

FIGS. 1A-1C illustrate (e.g., part of) a workflow for analyzing a biological sample comprising a mixture of biomolecules. The workflow may comprise an operation of contacting the biological sample with a surface (e.g., a particle, such as a nanoparticle as disclosed herein) to bind one or more proteins from among the plurality of biomolecules in the sample. Biological samples as disclosed herein may comprise complex mixtures of multiple biomolecules or classes of biomolecules, such as one or more proteins, lipids, and/or metabolites. In order to effectively identify or quantify biomolecules across diverse classes, the composition of the biological sample may be altered through one or more contacting operations to extract certain biomolecules or classes of biomolecules or to bind (e.g., adsorb) one or more biomolecules. By binding one or more biomolecules or classes of biomolecules, surfaces as disclosed herein may be used to isolate those biomolecules or classes of biomolecules for further downstream analysis or to reduce their concentration in a sample to permit extraction and analysis of other biomolecules and classes of biomolecules. In some cases, the workflow further comprises an operation of contacting the biological sample with a solvent system as disclosed herein. As illustrated in FIGS. 1A and 1B, the solvent system may be configured (e.g., by comprising one or more organic solvents as disclosed herein) to extract one or more lipids from the biological sample. The one or more lipids may partition into an organic phase which is relatively hydrophobic. As discussed herein throughout, the particular identity and relative composition of the solvent system may be adjusted to modulate the breadth of lipid classes (e.g., relatively more or fewer classes) extracted for downstream analysis. Solvent systems may additionally or alternatively be selected for their compatibility with lipidomic or multiomic workflows as detailed herein. The workflow may further comprise an operation of contacting the biological sample (or a portion thereof) with a surface as disclosed herein to interact with (e.g., adsorb or bind) one or more biomolecules in the sample. Additionally, or alternatively, the solvent system can comprise solvents to extract one or more polar metabolites from the biological sample. The one or more metabolites may partition into an aqueous (or other relatively polar) phase. The particular identity of the solvent system can be adjusted to modulate the breadth of metabolite classes (e.g., relatively more or relatively fewer classes) extracted for downstream analysis.

As illustrated in FIGS. 1A-1C, the surface may comprise a surface of a particle 102. Alternatively, the surface may comprise a substantially planar surface. As illustrated in FIG. 1A, the surface 102 may contact the biological sample 100 (e.g., plasma) to provide an altered composition of the biological sample 104. As illustrated in FIG. 1A, the altered composition of the biological sample 104 may be brought about by the interaction of one or more components (e.g., biomolecules, such as proteins, lipids, and/or metabolites) of the biological sample 100 with the surface 102 to produce surface with one or more biomolecules bound 106. As illustrated in FIG. 1A, the altered composition of the biological sample 104 and the surface with one or more bound biomolecules 106 may be separated and processed according to different workflows as described herein for further analysis. The separation may be performed by, for example, centrifugation (e.g., ultracentrifugation), magnetic separation, filtration, gravitational separation, solid phase extraction (e.g., column separation, such as spin-column separation), or any combination thereof. Alternatively, the altered composition of the biological sample 104 and the surface with one or more bound biomolecules 106 may be processed together. The organic solvent may then be contacted to the altered composition of the biological sample 104 in operation 101 to provide an organic layer 110 comprising one or more lipids for downstream processing and/or analysis. As illustrated in FIG. 1A, operation 101 may produce one or more additional layers that are optionally subjected to further analysis. FIG. 1A illustrates aqueous layer 112 and protein pellet 114 that may result from operation 101 and may optionally be subjected to further processing, analogous to organic layer 110. In an example, aqueous layer 112 is similarly isolated as with respect to organic layer 110 and aqueous layer 112 is subjected to further processing and/or analysis as described herein (e.g., to identify one or more polar metabolites).

The altered composition of the biological sample 104 may arise due to the types and quantities of biomolecules which interact with the surface 102. In an example (e.g., as depicted in FIG. 1A), the altered composition of the biological sample 104 comprises a reduced amount of at least one protein (e.g., when the surface 102 interacts with at least one protein present in the biological sample 100).

Alternatively, the biological sample 100 may be first contacted with the organic solvent in operation 101, and a portion of the biological sample such as the organic layer 110 or aqueous layer 112 contacted with the surface 102, as illustrated in FIG. 1B. For example, FIG. 1B illustrates separation of organic layer 110 from aqueous layer 112 and protein pellet 114 prior to contacting organic layer 110 with surface 102. Alternatively, or additionally, aqueous layer 112 may be contacted with surface 102. Following contacting with surface 102, organic layer 110 and surface with one or more biomolecules bound 106 may be separated for further processing and/or analysis as described herein.

In another example, such as that depicted in FIG. 1C, surface 102 may be configured to interact with a plurality of classes of biomolecules (e.g., proteins, lipids, and/or metabolites). In an example illustrated in FIG. 1C, biological sample 100 is contacted with surface 102 to produce altered composition of the biological sample 104. The altered composition of the biological sample 104 can comprise a reduced amount of one or more biomolecules which interact with surface 102 to produce surface with one or more biomolecules bound 106. As depicted in FIG. 1C, surface with one or more biomolecules bound 106 may be separated from altered composition of the biological sample 104 and at least a part of the bound biomolecules separated from surface 102 to provide composition 120 for further processing and analysis (e.g., identification and/or quantitation of one or more biomolecules). In some cases, the surface 102 is configured to interact with one or more proteins or classes of proteins. In some cases, the surface 102 is configured to interact with one or more lipids or classes of lipids. In some cases, the surface 102 is configured to interact with one or more polar metabolites or classes of polar metabolite. In some cases, the surface 102 is configured to interact with multiple classes of biomolecules, such as at least two of proteins, lipids, and metabolites. In some cases, the surface 102 is configured to interact with at least all of proteins, lipids, and metabolites.

The methods and systems as described herein may comprise contacting a biological sample with a plurality of surfaces. Pluralities of surfaces may comprise a plurality of distinct surface types, which interact with (e.g., adsorb or bind) distinct ensembles of biomolecules. The surface types of a plurality of surfaces may be varied in their physicochemical properties (e.g., size, surface charge, surface chemistry, porosity, morphology, and other properties as disclosed herein). In some cases, surface types may also share one or more of these physicochemical properties.

In some cases, the plurality of biomolecules comprises at least one metabolite. In some cases, the method comprises contacting the biological sample with a surface to bind the at least one protein, thereby yielding an altered composition of the biological sample. In some cases, the altered composition of the biological sample comprises a reduced amount of the at least one protein. In some cases, the method comprises adding a first solvent to the altered composition of the biological sample to generate a mixture comprising a first organic layer and an aqueous layer. In some cases, the at least one lipid or metabolite partitions into the first organic layer. In some cases, the method comprises performing mass spectrometry on at least a portion of the first organic layer, thereby identifying the at least one lipid or metabolite.

Methods as disclosed herein may further comprise an operation detecting or identifying a presence or absence of one or more of substances (e.g., biomolecules, such as proteins, lipids, or metabolites) in a sample. The substances may be detected by various methods or techniques as described herein. Non-limiting examples of detectors for use with the methods described herein may include flame ionization detector, aerosol-based detector, flame photometric detector, atomic-emission detector, nitrogen phosphorus detector (NPD), evaporative light scattering detector (ELSD), mass spectrometer (MS) (e.g., quadrupole MS, orthogonal MS, etc.), UV detectors (e.g., diode array detector (DAD or PDA)), thermal conductivity detector (TCD), fluorescence detector, electron capture detector (ECD), conductivity monitor, photoionization detector (PID), refractive index detector (RI or RID), radio flow detector, chiral detector, Raman spectrometer, nuclear magnetic resonance (NMR) spectrometer, UV-vis spectrometer, or combinations thereof.

Mass Spectrometry (MS) is an analytical technique that can be used for identifying the amount and type of chemicals present in a sample, determining the elemental composition of samples, quantitating the mass of particles and molecules, and elucidating the chemical structure of molecules by measuring the mass-to-charge ratio and the abundance of gas-phase ions. Various types of MS-based technologies with high specificity, such as liquid chromatography (LC-MS), gas chromatography (GC-MS), desorption electrospray ionization, and matrix-assisted laser desorption/ionization/time-of-flight (MALDI-TOF MS), can be utilized as part of the systems and methods described herein.

In cases where a MS detector is utilized, the presence or absence of the substances or substances may be detected based on their ionization patterns in the mass spectrometer. The ions are accelerated under vacuum in an electric field and separated by mass analyzers according to their m/z ratios. Representative mass analyzers for use with the methods and systems disclosed herein include triple-quadrupole, time-of-flight (TOF), magnetic sector, orbitrap, ion trap, quadrupole-TOF, matrix-assisted laser desorption ionization (MALDI), ion mobility, and Fourier transform ion cyclotron resonance (FTICR) analyzers, and the like.

Prior to undergoing one or more identification or quantification operations (e.g., mass spectrometry), biomolecules or fractions or portions of samples comprising biomolecules may undergo one or more processing operations. Such processing operations can include one or more purification, isolation, separation, digestion (e.g., enzymatic), identification, or quantification, operations, or any combination thereof. In some cases, a biomolecule (e.g., lipid) may be purified prior to an analysis (e.g., mass spectrometry) operation. In some cases, a biomolecule which is absorbed to a surface may be separated from the surface prior to analysis (e.g., mass spectrometry analysis).

### Extraction Solvents

Solvent systems for extraction for metabolomics and lipidomic analyses may employ organic solvents (e.g., dichloromethane (DCM), methyl tert-butyl ether (MTBE), or chloroform). However, in consideration of automation, the extraction solvents used may be problematic under certain conditions (Table 1). For example, in Folch extraction, chloroform or dichloromethane may be incompatible with plasticware (e.g., polypropylene (PP)); a chloroform layer forms a bottom layer which is difficult to extract cleanly; and the extraction must be well ventilated due to the toxicity of the solvent. MTBE may be compatible with PP, but it may still require ventilation due to toxicity and high volatility. The butanol/methanol biphasic extraction may be incompatible with plastic depending on the choice of second solvent (e.g., heptane or ethyl acetate) and may further require ventilation due to the volatility of the second solvent (e.g., heptane). The butanol/methanol single phase extraction is a simple method and compatible with plastics, but this method generally does not induce any phase separation.

TABLE 1
Characteristics of different solvent systems

| | | Methyl tert- | | |
|---|---|---|---|---|
| | | butyl ether | Butanol/methanol | Butanol/methanol |
| | | (MTBE) | biphasic | monophase |
| Categories | Folch extraction | extraction | extraction | extraction |
| Solvents | Chloroform or | MTBE | Butanol, heptane, | Butanol |
| | dichloromethane | | and ethyl acetate | |
| Toxicity | Inhalation | Inhalation | Some inhalation | Mild inhalation |
| | toxicity | toxicity | toxicity | toxicity |
| Evaporation | Fast evaporation | Fast evaporation | Unknown | Slow evaporation |
| | | | evaporation rate | |
| Approximate | Greater than 2 | Greater than 1.5 | Less than 1 mL | Less than 200 μL |
| Solvent | mL | mL | | |
| Volume | | | | |
| Polypropylene | Incompatible | Compatible with | Possibly | Compatible with |
| (PP) | with PP | PP | incompatible with | PP |
| compatibility | | | PP (heptane and | |
| | | | ethyl acetate) | |

To address these and other issues, in some aspects, the disclosure provides operations for performing liquid extractions, optionally as part of a lipidomics, metabolomics, or multiomics workflows. In some cases, the methods disclosed herein show competitive identification performance across hundreds of distinct molecular targets in the lipidome and metabolome. The extraction methods can be based on (e.g., comprise or comprise the use of) organic solvents (e.g., butyl acetate) which are not subject to the same drawbacks as some solvents or solvent systems discussed herein. In some cases, the extraction methods disclosed herein are more amenable to manual automated workflows. In some cases, the methods disclosed herein alleviate the need for toxic and/or highly volatile solvents, such as MTBE, DCM, or chloroform. In some cases, the solvents exhibit reduced vapor pressure and/or viscosity, preventing imprecision in liquid handling.

In some aspects, the methods and systems disclosed herein comprise an operation of performing a liquid extraction (e.g., contacting a biological sample or portion thereof with a liquid solvent). In some cases, the liquid extraction comprises forming multiple layers or phases of liquid solvents to extract and separated certain compounds or classes of compounds. An example extraction operation 101 is illustrated in FIG. 1D. A biological sample 100 is provided. The biological sample in FIG. 1D is depicted as comprising about 10-100 μL of plasma, though any biological sample as disclosed herein may be used with the extraction operation(s) illustrated in FIG. 1D. At step 1011, an organic solvent (and optionally water, as illustrated in FIG. 1D) is added to the biological sample 100 and optionally centrifuged or otherwise agitated or mixed to provide composition phase-separated composition 1002. Phase-separated composition 1002 is depicted as comprising an organic layer comprising lipid(s) present in the starting biological sample 100. Phase-separated composition 1002 is also depicted as comprising a water layer comprising water present in the starting biological sample 100 as well as water added during step 1001 and polar metabolites that substantially partition into the aqueous layer. However, in liquid extractions where water is not added, the water layer may not form. Phase-separated composition 1002 is also depicted as comprising a protein pellet comprising proteinaceous material which precipitates out of solution during the extraction. As illustrated in FIG. 1D, the organic layer or phase-separated composition 1002 is above the aqueous layer. In some cases, (e.g., when the organic layer has a density greater than a density of water), the organic layer may be below the aqueous layer. At step 1013, the separated phases of phase-separated composition 1002 are transferred to separate vessels to provide an organic layer 1004 and an aqueous layer 1006. Each layer may separately be provided for further downstream analysis. In the example depicted in FIG. 1D, organic layer 1004 and aqueous layer 1006 are separately dried down and sent off for downstream analysis, such as liquid chromatography-mass spectrometry. In some cases, only one phase may be analyzed. In some cases, the analysis comprises an affinity-based, spectrographic, or other assay as disclosed herein. The choice of liquid chromatography (or other downstream separation operation) may be determined by the identity or predicted identity of one or more biomolecules in a given phase. For example, reverse phase liquid chromatography (RPLC) may be used as shown in FIG. 1D to separate biomolecules comprised in the organic phase. In another example, hydrophobic interaction chromatography (HILIC) may be used to separate biomolecules (e.g., metabolites) comprised in the aqueous layer, as shown in FIG. 1D.

As illustrated in FIG. 1D, certain biomolecules or classes of biomolecules may preferentially partition into corresponding layers based on the complementary non-covalent interactions between those biomolecules and the particular solvent(s) used. For example, a relatively nonpolar (or hydrophobic) solvent may extract relatively nonpolar classes of biomolecules (e.g., lipids) while a more polar solvent (e.g., water) may extract relatively polar classes of biomolecules (e.g., polar metabolites). Based on the properties of various solvents disclosed herein, a liquid extraction workflow may comprise one or more solvents configured to separate or isolate target biomolecules or classes thereof from a biological sample.

In some cases, the organic solvent added at step 1011 may comprise a plurality of organic solvents. For example, the plurality or organic solvents may comprise one or more alcohols (e.g., methanol, ethanol, butanol) and a second, relatively more non-polar solvent (e.g., alkane, ether, ester) as disclosed herein. The plurality of organic solvents may be added in any order or simultaneously. Further, additional solvents may be added to only a subset of phases from a previous round of extraction. For example, a first solvent may be added to a biological sample which causes the sample to partition into two layers (e.g., organic and aqueous). The second solvent may subsequently be added to the mixture of the two layers, or the organic and aqueous layers may be separated, and the second solvent may be added to one or the other. Further, any number of extractions with solvents as described herein may be performed. Solvent systems may comprise individual solvents in any proportion, such as those disclosed herein.

Solvents or solvent systems may be selected on the basis of their measured ability to extract one or more biomolecules or of biomolecules from samples. On the basis of their chemical properties, solvents may preferentially interact with and thus extract certain biomolecules or classes of biomolecules over others. Further, certain solvents may extract certain biomolecules or classes of biomolecules in a manner that is more reproducible than other solvents or solvent systems. Reproducibility may be measured by performing an extraction as described herein using substantially the same solvent or solvent system across a plurality of biological samples or plurality of replicates of the same biological sample and comparing the amounts (e.g., relative or absolute) of extracted biomolecules subsequently detected. In an example, a plurality of biological samples as described herein is extracted using a solvent system comprising butyl acetate. The extracted biomolecules are subjected to identification and quantification (e.g., by mass spectrometry), and coefficients of variation are calculated for a subset of detected biomolecules or fragments thereof. As compared to extractions of the same solvent system comprising another solvent system, the extraction with butyl acetate results in overall lower individual coefficients of variation or a lower median, mean, or standard deviation of a distribution of such coefficients of variation. In some cases, an extraction with a solvent as described herein results in overall lower individual coefficients of variation or a lower median, mean, or standard deviation of a distribution of such coefficients of variation as compared to an extraction performed using a reference solvent. In some cases, the reference solvent comprises DCM, chloroform, MTBE, methanol, butanol, ethyl acetate, or any combination thereof.

Alternatively, or additionally, reproducibility may be assessed by performing multiple replicates of an extraction as described herein. Vectors of features as described herein corresponding to each replicate are subjected to a dimensionality reduction algorithm (e.g., principal component analysis (PCA)) to project the data into a lower dimension space. Components are ranked (1 to n, where n is the total number of components) in order of highest variance explained by the data to the lowest. Replicates are plotted in this lower dimension space. For solvents which extract with higher reproducibility, the corresponding replicates are found to cluster close to one another in the lower dimensional space. Furthermore, the mutual distance between the replicates within the cluster may be smaller than the distances between replicates in other clusters corresponding to other extraction solvents. In some cases, an extraction with a solvent as described herein results in a lower apparent variability when the results of identification and/or quantification of the extracted biomolecules are analyzed by PCA as compared to a reference solvent. In some cases, the reference solvent comprises DCM, chloroform, MTBE, methanol, butanol, ethyl acetate, or any combination thereof.

In addition to being selected on the basis of relative polarity, solvents for liquid extractions as disclosed herein may be selected on the basis of any other chemical or physical property. In particular, solvents or combinations of solvents may be selected which display physical properties that are compatible with (e.g., automated) omics workflows as described herein. In some cases, a solvent comprises a certain density (e.g., higher or lower than water at a density of water). In an example, a solvent is used which has a density less than a density of water. In such cases, the solvent may partition to the top of the phase-separated composition, facilitating isolation or the organic layer. In some cases, a solvent comprises a certain dielectric constant (e.g., higher or lower than a dielectric constant of water). In an example, the solvent comprises a dielectric constant less than water. In such cases, the solvent preferentially isolates nonpolar biomolecules (e.g., lipids) which are the target of downstream analysis. In some cases, a solvent comprises a certain viscosity (e.g., above or below a threshold viscosity). In an example, a solvent comprises a viscosity above a threshold. In such cases, the solvent is sufficiently viscous and cohesive that it does not drip out of instrumentation (e.g., a pipette) during a workflow as described herein. In some cases, a solvent comprises or a certain vapor pressure (e.g., above or below a threshold vapor pressure). In an example, a solvent has a vapor pressure below a threshold. In such cases, the solvent is sufficiently nonvolatile that extraordinary safety precautions are not required when using the solvent in a workflow described herein. In some cases, a solvent displays any combination of these properties.

In some cases, a solvent comprises an alcohol, an aliphatic hydrocarbon, an aromatic hydrocarbon, an aliphatic heterocyclic compound, an aromatic heterocyclic compound, an amide, an ester, an ether, a ketone, a halocarbon, or a nitrile, or any combination thereof. In some cases, a solvent comprises an alcohol. In some cases, a solvent comprises an aliphatic hydrocarbon. In some cases, a solvent comprises an aromatic hydrocarbon. In some cases, a solvent comprises an aliphatic heterocyclic compound. In some cases, a solvent comprises an aromatic heterocyclic compound. In some cases, a solvent comprises an amide. In some cases, a solvent comprises an ester. In some cases, a solvent comprises an ether. In some cases, a solvent comprises a ketone. In some cases, a solvent comprises a halocarbon. In some cases, a solvent comprises a nitrile. In some cases, a solvent does not comprise a halocarbon (e.g., chloroform, DCM).

In some cases, a solvent comprises an ester. In some cases, a solvent comprises methyl formate. In some cases, the solvent comprises methyl acetate. In some cases, the solvent comprises methyl propionate. In some cases, a solvent comprises methyl butyrate. In some cases, a solvent comprises methyl valerate. In some cases, a solvent comprises methyl hexanoate. In some cases, a solvent comprises ethyl formate. In some cases, a solvent comprises ethyl acetate. In some cases, a solvent comprises ethyl propionate. In some cases, a solvent comprises ethyl butyrate. In some cases, a solvent comprises ethyl valerate. In some cases, a solvent comprises ethyl hexanoate. In some cases, a solvent comprises propyl formate. In some cases, a solvent comprises propyl acetate. In some cases, a solvent comprises propyl propionate. In some cases, a solvent comprises propyl butyrate. In some cases, a solvent comprises propyl valerate. In some cases, a solvent comprises propyl hexanoate. In some cases, a solvent comprises butyl formate. In some cases, a solvent comprises butyl acetate. In some cases, a solvent comprises butyl propionate. In some cases, a solvent comprises butyl butyrate. In some cases, a solvent comprises butyl valerate. In some cases, a solvent comprises butyl hexanoate. In some cases, a solvent comprises pentyl formate. In some cases, a solvent comprises pentyl acetate. In some cases, a solvent comprises pentyl propionate. In some cases, a solvent comprises pentyl butyrate. In some cases, a solvent comprises pentyl valerate. In some cases, a solvent comprises pentyl hexanoate. In some cases, a solvent comprises hexyl formate. In some cases, a solvent comprises hexyl acetate. In some cases, a solvent comprises hexyl propionate. In some cases, a solvent comprises hexyl butyrate. In some cases, a solvent comprises hexyl valerate. In some cases, a solvent comprises hexyl hexanoate. In some cases, a solvent comprises any combination or isomer of any of these esters.

In some cases, a solvent comprises diethyl ether, methyl tert-butyl ether (MTBE), dioxane, tetrahydrofuran (THF), or anisole, or any isomer or combination thereof. In some cases, a solvent comprises diethyl ether or any isomer thereof. In some cases, a solvent comprises MTBE or any isomer thereof. In some cases, a solvent comprises dioxane or any isomer thereof. In some cases, a solvent comprises THF or any isomer thereof. In some cases, a solvent comprises anisole or any isomer thereof.

In some cases, a solvent comprises an alcohol. In some cases, a solvent comprises methanol, ethanol, propanol, butanol, pentanol, or hexanol, or any isomer or combination thereof. In some cases, a solvent comprises methanol or any isomer thereof. In some cases, a solvent comprises ethanol or any isomer thereof. In some cases, a solvent comprises propanol or any isomer thereof. In some cases, a solvent comprises butanol or any isomer thereof. In some cases, a solvent comprises pentanol or any isomer thereof. In some cases, a solvent comprises hexanol or any isomer thereof.

A solvent may comprise a binary mixture of solvents in any amount. In some cases, a solvent comprises a mixture of two solvents comprising a volume/volume (v/v) ratio of at least about 1:100, 1:90, 1:80, 1:70, 1:60, 1:50, 1:40, 1:30, 1:20, 1:10, 1:9. 1:8. 1:7. 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or more. In some cases, a solvent comprises a mixture of two solvents comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some cases, a solvent comprises a mixture of two solvents comprising a volume/volume (v/v) ratio of no more than about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30. 1:40, 1:50, 1:60. 1:70, 1:80, 1:90:1:100, or less. In some cases, a solvent comprises a mixture of two solvents comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some cases, a solvent comprises a mixture of butanol and methanol comprising a volume/volume (v/v) ratio of at least about 1:100, 1:90, 1:80, 1:70, 1:60, 1:50, 1:40, 1:30, 1:20, 1:10, 1:9. 1:8. 1:7. 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or more. In some cases, a solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some cases, a solvent comprises a mixture of butanol:methanol comprising a volume/volume (v/v) ratio of no more than about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30. 1:40, 1:50, 1:60. 1:70, 1:80, 1:90:1:100, or less. In some cases, a solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some cases, a solvent comprises a mixture of about 3, 4, 5, 6, 7, 8, 9, 10, 11, 12, 13, 14, 15, 16, 17, 18, 19, or 20, or more solvents. In some cases, a solvent comprises a mixture of at least about 3, 4, 5, 6, 7, 8, 9, 10, 11, 12, 13, 14, 15, 16, 17, 18, 19, or 20, or more solvents. In some cases, a solvent comprises a mixture of no more than about 20, 19, 18, 17, 16, 15, 14, 13, 12, 11, 10, 9, 8, 7, 6, 5, 4, 3, or 2 solvents. The mixture of solvents may comprise any relative proportion of each constituent solvent.

In some cases, a density of a solvent is lower than a density of water. In some cases, a density of a solvent is higher than a density of water. In some cases, a density of a solvent is equal to a density of water.

In some cases, a dielectric constant of a solvent at room temperature is lower than a dielectric constant of water. In some cases, the dielectric constant of a solvent is higher than the dielectric constant of water. In some cases, the dielectric constant of a solvent is equal to the dielectric constant of water. In some cases, a dielectric constant of a solvent is less than about 1, about 2, about 3, about 4, about 5, about 6, about 7, about 8, about 9, about 10, about 20, about 30, about 40, about 50, about 60, about 70, or about 80 at about 20° C. In some cases, a dielectric constant of a solvent is more than about 1, about 2, about 3, about 4, about 5, about 6, about 7, about 8, about 9, about 10, about 20, about 30, about 40, about 50, about 60, about 70, or about 80 at about 20° C.

In some cases, a dielectric constant of a solvent at about 20° C. ranges from about 1 to about 80. In some cases, a dielectric constant of a solvent at about 20° C. ranges from about 1 to about 5, about 1 to about 10, about 1 to about 20, about 1 to about 30, about 1 to about 40, about 1 to about 50, about 1 to about 60, about 1 to about 70, about 1 to about 80, about 5 to about 10, about 5 to about 20, about 5 to about 30, about 5 to about 40, about 5 to about 50, about 5 to about 60, about 5 to about 70, about 5 to about 80, about 10 to about 20, about 10 to about 30, about 10 to about 40, about 10 to about 50, about 10 to about 60, about 10 to about 70, about 10 to about 80, about 20 to about 30, about 20 to about 40, about 20 to about 50, about 20 to about 60, about 20 to about 70, about 20 to about 80, about 30 to about 40, about 30 to about 50, about 30 to about 60, about 30 to about 70, about 30 to about 80, about 40 to about 50, about 40 to about 60, about 40 to about 70, about 40 to about 80, about 50 to about 60, about 50 to about 70, about 50 to about 80, about 60 to about 70, about 60 to about 80, or about 70 to about 80. In some cases, a dielectric constant of a solvent at about 20° C. ranges from about 1, about 5, about 10, about 20, about 30, about 40, about 50, about 60, about 70, or about 80. In some cases, a dielectric constant of a solvent at about 20° C. ranges from at least about 1, about 5, about 10, about 20, about 30, about 40, about 50, about 60, or about 70. In some cases, a dielectric constant of a solvent at about 20° C. ranges from at most about 5, about 10, about 20, about 30, about 40, about 50, about 60, about 70, or about 80.

In some cases, a viscosity of a solvent is greater than about 0.1 mPa·s, 0.15 mPa·s, 0.2 mPa·s, 0.25 mPa·s, 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, 0.8 mPa·s, 0.85 mPa·s, 0.9 mPa·s, or 0.95 mPa·s at about 20° C. In some cases, a viscosity of a solvent is greater than about 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, or 0.8 mPa·s. In some cases, a viscosity of a solvent is about 0.1 mPa·s, 0.15 mPa·s, 0.2 mPa·s, 0.25 mPa·s, 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, 0.8 mPa·s, 0.85 mPa·s, 0.9 mPa·s or 0.95 mPa·s at about 20° C. In some cases, a viscosity of a solvent is about 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, or 0.8 mPa·s.

In some cases, a vapor pressure of a solvent is less than about 600, about 550, about 500, about 450, about 400, about 350, about 300, about 250, about 200, about 150, about 100, about 90, about 80, about 70, about 60, about 50, about 40, or about 30 torr at about 20° C. In some cases, a vapor pressure of a solvent is less than about 400, about 300, about 250, about 200, about 100, about 90, about 80, about 70, about 60, or about 50 torr at about 20° C. In some cases, a vapor pressure of a solvent is about 600, about 550, about 500, about 450, about 400, about 350, about 300, about 250, about 200, about 150, about 100, about 90, about 80, about 70, about 60, about 50, about 40, or about 30 torr at about 20° C. In some cases, a vapor pressure of a solvent is about 400, about 300, about 250, about 200, about 100, about 90, about 80, about 70, about 60, or about 50 torr at about 20° C.

In some cases, the methods disclosed herein further comprise adding a second solvent to the altered composition of the biological sample, the first organic layer, or the aqueous layer. The second solvent may comprise any solvent or a solvent with any property as disclosed herein. In some cases, the methods disclosed herein further comprise adding a second solvent to the altered composition of the biological sample. In some cases, the methods disclosed herein further comprise adding a second solvent to the first organic layer. In some cases, the methods disclosed herein further comprise adding a second solvent to the aqueous layer. In some cases, the second solvent substantially partitions into the first organic layer. In some cases, the second solvent substantially partitions into a second organic layer.

In some cases, the first solvent comprises an alcohol and the second solvent comprises an ester. In some cases, the first solvent comprises an alcohol. In some cases, the alcohol comprises butanol, methanol, or a combination thereof. In some cases, the alcohol comprises butanol. In some cases, the alcohol comprises methanol. In some cases, the second solvent comprises an ester. In some cases, the ester comprises butyl acetate.

In some cases, the first solvent comprises an alcohol and the second solvent comprises an ester and an ether. In some cases, the first solvent comprises an alcohol. In some cases, the alcohol comprises butanol, methanol, or a combination thereof. In some cases, the alcohol comprises butanol. In some cases, the alcohol comprises methanol. In some cases, the second solvent comprises an ester and an ether. In some cases, the second solvent comprises an ester. In some cases, the ester comprises butyl acetate. In some cases, the second solvent comprises an ether. In some cases, the ether comprises methyl tert-butyl ether (MTBE). In some cases, the methods disclosed herein further comprise adding a second solvent to the biological sample, the first organic layer, or the aqueous layer. In some cases, the methods disclosed herein further comprise adding a second solvent to the biological sample. In some cases, the methods disclosed herein further comprise adding a second solvent to the first organic layer. In some cases, the methods disclosed herein further comprise adding a second solvent to the aqueous layer.

In some cases, the second solvent substantially partitions into the first organic layer. In some cases, the second solvent substantially partitions into a second organic layer.

In some cases, the second solvent comprises an alcohol. In some cases, the second solvent comprises methanol, ethanol, propanol, butanol, pentanol, or hexanol, or any isomer or combination thereof. In some cases, the second solvent comprises methanol or any isomer thereof. In some cases, the second solvent comprises ethanol or any isomer thereof. In some cases, the second solvent comprises propanol or any isomer thereof. In some cases, the second solvent comprises butanol or any isomer thereof. In some cases, the second solvent comprises pentanol or any isomer thereof. In some cases, the second solvent comprises hexanol or any isomer thereof.

In some cases, the second solvent comprises a mixture of two solvents comprising a volume/volume (v/v) ratio of at least about 1:100, 1:90, 1:80, 1:70, 1:60, 1:50, 1:40, 1:30, 1:20, 1:10, 1:9. 1:8. 1:7. 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or more. In some cases, the second solvent comprises a mixture of two solvents comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some cases, the second solvent comprises a mixture of two solvents comprising a volume/volume (v/v) ratio of no more than about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30. 1:40, 1:50, 1:60. 1:70, 1:80, 1:90:1:100, or less. In some cases, the second solvent comprises a mixture of two solvents comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some cases, the second solvent comprises a mixture of butanol and methanol comprising a volume/volume (v/v) ratio of at least about 1:100, 1:90, 1:80, 1:70, 1:60, 1:50, 1:40, 1:30, 1:20, 1:10, 1:9. 1:8. 1:7. 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, or more. In some cases, the second solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. In some cases, the second solvent comprises a mixture of butanol and methanol comprising a volume/volume (v/v) ratio of no more than about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30. 1:40, 1:50, 1:60. 1:70, 1:80, 1:90:1:100, or less. In some cases, the second solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. In some cases, a second solvent comprises a mixture of about 3, 4, 5, 6, 7, 8, 9, 10, 11, 12, 13, 14, 15, 16, 17, 18, 19, or 20, or more solvents. In some cases, a second solvent comprises a mixture of at least about 3, 4, 5, 6, 7, 8, 9, 10, 11, 12, 13, 14, 15, 16, 17, 18, 19, or 20, or more solvents. In some cases, a solvent comprises a mixture of no more than about 20, 19, 18, 17, 16, 15, 14, 13, 12, 11, 10, 9, 8, 7, 6, 5, 4, 3, or 2 solvents. The mixture of solvents may comprise any relative proportion of each constituent solvent.

In some cases, the second solvent comprises an ester and an ether. In some cases, the second solvent comprises an ester. In some cases, the ester comprises butyl acetate. In some cases, the second solvent comprises an ether. In some cases, the methyl tert-butyl ether (MTBE).

### Biomolecule Adsorption

Methods and systems as described herein may comprise an operation of contacting a biological sample or part thereof with a surface to adsorb (e.g., bind) one or more biomolecules or sets of biomolecules. The surfaces may be configured to (e.g., comprise certain physiochemical properties as described herein) adsorb certain biomolecules or classes of biomolecules, thereby providing an altered composition of the biological sample or portion thereof. In some cases, (e.g., as part of a proteomics arm of a multiomics workflow as described herein), the biomolecules adsorbed to the surface(s) may be further analyzed as described elsewhere herein. In some cases, the surface(s) may adsorb biomolecules to alter a composition of a biological sample which is further processed (e.g., by a liquid extraction operation as discussed above) to isolate other biomolecules or classes of biomolecules (e.g., lipids, metabolites) for downstream analysis.

Methods and systems as disclosed herein may comprise contacting any fraction or derivative of a biological sample with the surface. For example, as discussed above, the biological sample may be contacted directly with the surface. In some cases, an organic layer (e.g., generated by contacting a sample with an organic solvent as discussed above) may be contacted with the surface. In some cases, an aqueous layer may be contacted with a surface. In some cases, the aqueous layer and the organic layer may be contacted with the surface.

Methods and systems as disclosed herein may comprise contacting multiple samples or portions thereof with surface. In an example, the organic layer may be contacted with a first surface and the aqueous layer may be contacted with a second surface. In another example, the biological sample is contacted with a first surface and an organic layer generated by a liquid extraction step is contacted with a second surface. In some cases, contacting different components or derivatives of a biological sample with the same or different surface may allow for additional biomolecules to be adsorbed and detected downstream. In some cases, contacting different components with the same or different surface may allow for additional biomolecules to be adsorbed, thereby restricting the set of biomolecules which is detected downstream

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

In some cases, non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle, or at most 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules, proteins, lipids, or metabolites may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules, proteins, lipids, or metabolites may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules, proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules, proteins, lipids, or metabolites may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules, proteins, lipids, or metabolites may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules, proteins, lipids, or metabolites may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules, proteins, lipids, or metabolites may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules, proteins, lipids, or metabolites may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules, proteins, lipids, or metabolites may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules, proteins, lipids, or metabolites may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins, lipids, or metabolites. In some cases, adsorbed proteins, lipids, or metabolites may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins, lipids, or metabolites. In some cases, adsorbed proteins, lipids, or metabolites may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins, lipids, or metabolites.

In some cases, proteins, lipids, or metabolites in a biological sample may span at least about 1, 2, 3, 4, 5, 6, 7, 8, 9, 10, 15, 20, 25, or 30, or more, orders of magnitudes in concentration. In some cases, proteins, lipids, or metabolites in a biological sample may span at most about 1, 2, 3, 4, 5, 6, 7, 8, 9, 10, 15, 20, 25, or 30, or more, orders of magnitudes in concentration.

As used herein, “lipidomic analysis,” “lipids analysis,” and the like, generally refer to any system or method for analyzing lipids in a sample, including the systems and methods disclosed herein. Also, as used herein, “metabolomic analysis,” “metabolites analysis,” and the like, generally refer to any system or method for analyzing metabolites in a sample, including the systems and methods disclosed herein. Also, “proteomic analysis,” “proteins analysis,” and the like, generally refer to any system or method for analyzing proteins in a sample, including the systems and methods disclosed herein.

Systems and methods of the present disclosure for assaying a biological sample may comprise an operation of contacting a biological sample or part, fraction, or derivative thereof with one or more surfaces. In some cases, a surface may comprise a surface of a high surface-area material, such as nanoparticles, particles, microparticles, or porous materials. As used herein, a “surface” generally refers to a surface for assaying biological molecules or derivatives or fragments thereof, such as proteins, amino acid, poly amino acid, lipids, fatty acids, and small molecule metabolites. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, lipids, lipid concentrations, metabolite (e.g., non-proteinaceous small molecule metabolites) or any combination thereof. In some cases, polyamino acids in the sample adsorb to distinct surfaces to form one or more adsorption layers of biomolecules. In some cases, nucleic acids in the sample adsorb to the distinct surfaces to form one or more adsorption layers of biomolecules. In some cases, lipids or fragments thereof, such as fatty acids, in the sample adsorb to the distinct surfaces to form one or more adsorption layers of biomolecules. In some cases, small molecule metabolites in the sample adsorb to the distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., lipids, metabolites). In some cases, the enriching may be selective—e.g., biomolecules in the subset may be enriched but biomolecules outside of the subset may not enriched and/or be depleted. In some cases, a panel including any number of distinct particle types disclosed herein, may enrich and identify biomolecules over a dynamic range of at least 5, 6, 7, 8, 9, 10, 11, 12, 13, 14, 15, or 20 orders of magnitude. In some cases, a panel including any number of distinct particle types disclosed herein, may enrich and identify biomolecules over a dynamic range of at most 5, 6, 7, 8, 9, 10, 11, 12, 13, 14, 15, or 20 orders of magnitude.

A panel can have more than one surface type. Increasing the number of surface types in a panel can be a method for increasing the number of biomolecules that can be identified in a given sample. In some cases, increasing the number of surface types can further focus an analysis by removing extraneous or undesired biomolecules from a sample.

A surface (e.g., particle) may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dipalmitoylphosphatidylcholine (DPPC), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, phosphatidic acid, dicetylphosphate, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a poly(N-[3-(dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some cases, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some cases, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalization, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a binding molecule. The binding molecule may be a small molecule, an oligomer, or a macromolecule. The binding molecule may comprise a binding specificity for a group or class of analytes (e.g., a plurality of saccharides or a class of proteins). A binding molecule may comprise a moderate binding specificity for the group or class of analytes. Conversely, a binding molecule may comprise a dis-affinity for a group or class of analytes, disfavoring binding of these species relative to the same particle lacking the binding molecule. For example, a binding molecule may comprise a negative charge distribution which repels negatively charged nucleic acids, thereby disfavoring their binding.

A binding molecule may comprise a peptide. Peptides are an extensive and diverse set of biomolecules which may comprise a wide range of physical and chemical properties. Depending on its composition, sequence, and chemical modification, a peptide may be hydrophilic, hydrophobic, amphiphilic, lipophilic, lipophobic, positively charged, negatively charged, zwitterionic, neutral, chaotropic, antichaotropic, reactive, redox active, inert, acidic, basic, rigid, flexible, or any combination thereof. Accordingly, a peptide surface functionalization may confer a range of physicochemical properties to a particle. A particle may comprise a single peptide surface functionalization or a plurality of peptide surface functionalizations. A single peptide surface functionalization may comprise a plurality of identical or sequence-sharing peptides bound to a particle in a uniform fashion.

A surface functionalization may comprise a small molecule functionalization, a macromolecular functionalization, or a combination of two or more such functionalization. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, A surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some embodiments, a macromolecule can be modified with a peptide. In some embodiments, the macromolecule comprises a thiol or azide. In some embodiments, a surface comprises the macromolecule modified with a peptide immobilized to a surface. In some embodiments, the macromolecule is covalently coupled to the surface. In some embodiments, the macromolecule is electrostatically coupled to the surface. In some embodiments, the macromolecule is coupled to the surface through a polymerization event. In some embodiments, the polymerization event comprises a reaction with a vinyl group on the surface.

In some embodiments, macromolecules modified with peptides can be immobilized on surfaces for identification, binding, or enrichment of biomolecules (e.g., proteins). In some embodiments, a surface can comprise a macromolecule modified with a peptide, wherein the peptide comprises a binding site, and a protein interacting with the peptide at the binding site. In some embodiments, a biological sample can be contacted with a surface comprising the macromolecule modified with a peptide, wherein the peptides are configured to bind to a protein, which can release the plurality of biomolecules from the surface.

In some cases, the particle is a micelle, liposome, iron oxide particle, silver particle, gold particle, palladium particle, quantum dot, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, 2-(3-aminopropylamino) ethanol particle, protein functionalized particle, ubiquitin functionalized particle, polysaccharide coated particle, or dextran functionalized particle.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties.

A particle of the present disclosure may be a nanoparticle. A nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. A microparticle may be less than 1000 μm in diameter. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, a particle may comprise a diameter of at least about 10, 20, 30, 40, 50, 60, 70, 80, 90, 100, 200, 300, 400, 500, 600, 700, 800, 900, or 1000 nm. In some cases, a particle may comprise a diameter of at most about 10, 20, 30, 40, 50, 60, 70, 80, 90, 100, 200, 300, 400, 500, 600, 700, 800, 900, or 1000 nm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 nm. In some cases, a particle may comprise a diameter of at least about 10, 20, 30, 40, 50, 60, 70, 80, 90, 100, 200, 300, 400, 500, 600, 700, 800, 900, or 1000 mm. In some cases, a particle may comprise a diameter of at most about 10, 20, 30, 40, 50, 60, 70, 80, 90, 100, 200, 300, 400, 500, 600, 700, 800, 900, or 1000 mm. In some cases, a particle may comprise a diameter of about 10 to 1000, 20 to 900, 30 to 800, 40 to 700, 50 to 600, 60 to 500, 70 to 400, 80 to 300, 90 to 200, or 100 to 1000 mm.

In some cases, a first size of a first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1000 times a second size of a second particle. In some cases, a first size of a first particle is about 1.1 to 1000, 1.2 to 900, 1.3 to 800, 1.4 to 700, 1.5 to 600, 1.6 to 500, 1.7 to 400, 1.8 to 300, 1.9 to 200, 2 to 100, 3 to 90, 4 to 80, 5 to 70, 10 to 60, 20 to 50, or 30 to 40 times a second size of a second particle. In some cases, a size of a first particle is within +40% of a size of a second particle, a size of a first particle is within +30% of a size of a second particle, a size of a first particle is within +25% of a size of a second particle, a size of a first particle is within +20% of a size of a second particle, a size of a first particle is within +15% of a size of a second particle, or a size of a first particle is within +10% of a size of a second particle. In some cases, a size of a first particle is within at least about +10%, +15%, +20%, +25%, +30%, +35%, +40% of a size of a second particle. In some cases, a size of a first particle is within at most about +10%, +15%, +20%, +25%, +30%, +35%, +40% of a size of a second particle. In some cases, a size of a first particle is within about +10% to +40%, +15% to +35%, +20% to +30%, +25% to +40% of a size of a second particle. In some cases, the first size is a first diameter, and the second size is a second diameter. In some cases, the first size is a first average size, and the second size is a second average size. In some cases, the first average size and the second average size are mean sizes or median sizes.

In some cases, a particle panel comprises at least about 1, 2, 3, 4, 5, 6, 7, 8, 9, or 10 parts of a first particle to about 1 part of a second particle. In some cases, a particle panel comprises at most about 1, 2, 3, 4, 5, 6, 7, 8, 9, or 10 parts of a first particle to about 1 part of a second particle. In some cases, a particle panel comprises about 1 to 10, 2 to 9, 3 to 8, 4 to 7, 5 to 6 parts of a first particle to about 1 part of a second particle. In some cases, a particle panel comprises about 15 parts of a first particle to about 6 parts of a second particle. In some cases, the parts are parts by weight, parts by volume, or parts by surface area. In some cases, the parts are parts by weight.

In some cases, a particle may be contacted with a biological sample (e.g., a biofluid, such as plasma or serum) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins, lipids, or metabolites) in a biological sample (e.g., a biofluid). In some cases, a surface disclosed herein may be incubated with a biological sample to adsorb at least about 1000, 2000, 3000, 4000, 5000, 6000, 7000, 8000, 9000, 10000, or more unique biomolecules. In some cases, a surface disclosed herein may be incubated with a biological sample to adsorb at most about 1000, 2000, 3000, 4000, 5000, 6000, 7000, 8000, 9000, or 10000, or more, unique biomolecules. In some cases, a surface disclosed herein may be incubated with a biological sample to adsorb at least about 1000, 2000, 3000, 4000, 5000, 6000, 7000, 8000, 9000, or 10000, or more, unique biomolecule groups. In some cases, a surface disclosed herein may be incubated with a biological sample to adsorb at most about 1000, 2000, 3000, 4000, 5000, 6000, 7000, 8000, 9000, or 10000, or more, unique biomolecule groups. In some cases, several different types of surfaces can be used, separately or in combination, to identify large numbers of biomolecules in a particular biological sample. In other words, surfaces can be multiplexed in order to bind and identify large numbers of biomolecules in a biological sample.

In some cases, surfaces may be multiplex to identify one type of biomolecule. In some cases, the biomolecule comprises a lipid. In some cases, the biomolecule comprises a protein. In some cases, the biomolecule comprises a metabolite. In some cases, surfaces may be multiplexed to identify multiple classes of biomolecules. In some cases, surfaces mare multiplex to identify any two of proteins, lipids, and polar metabolites. In some cases, surfaces mare multiplex to identify all three of proteins, lipids, and polar metabolites.

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound biomolecules present in the spatially isolated samples by placing the entire plate on a magnet. In some cases, this simultaneously pulls down the superparamagnetic particles in the particle panel. In some cases, the supernatant in each sample can be removed to remove the unbound biomolecules. In some cases, these steps (incubate, pull down) can be repeated to effectively wash the particles, thus removing residual background unbound biomolecules that may be present in a sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 unique proteins. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 unique proteins. In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 protein groups. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 protein groups. In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1,000,000 peptides. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1,000,000 peptides. In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, the panels disclosed herein can be used to identify a number of lipids, lipid fragments, lipid adducts, or lipid classes using a lipid analysis workflow. In some cases, lipid analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). In some cases, lipid analysis may comprise contacting a sample to distinct surface types (e.g., a particle panel), forming adsorbed biomolecule layers on the distinct surface types to provide an altered composition of the biological sample (e.g., depleted or enriched for a biomolecule or class of biomolecule, such as proteins). In order to further isolate specific classes of lipids, one or more liquid extraction operations (e.g., contacting the altered composition of the biological sample with an organic solvent) as described herein may be performed to provide an organic layer comprising one or more lipids, and the lipid analysis may comprise identifying lipids in the organic layer (e.g., by mass spectrometry). Performing mass spectrometry on a lipid may produce one or more feature intensities on a plot of mass to charge ratio versus intensity from a mass spectrometry run of a sample comprising the lipid. In some cases, these features can correspond to variably ionized fragments of lipids and/or fragments or adducts thereof. In some cases, using the data analysis methods described herein, feature intensities can be sorted into lipid groups. In some cases, performing mass spectrometry identifies at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000, or more distinct lipids. In some cases, performing mass spectrometry identifies at most about 100,000, 50,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids.

In some cases, performing mass spectrometry comprises identifying at least about 1, 2, 3, 4, 5, 6, 7, 8, 9, 10, 20, 30, 40, 50, 60, 70, 80, 90, 100, 500, 1,000, 5,000, 10,000, or more cholesteryl esters (ChEs) in the biological sample. In some cases, performing mass spectrometry comprises identifying at least about 10, 20, 30, 40, 50, 60, 70, 80, 90, 100, or more ChEs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 500, 1,000, 5,000, 10,000, or more ceramides (CERs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, or more CERs in the biological sample. In some cases, performing mass spectrometry comprises identifying at least about 1, 2, 3, 4, 5, 6, 7, 8, 9, 10, 20, 30, 40, 50, 60, 70, 80, 90, 100, 500, 1,000, or more cholesterols (Chs) in the biological sample. In some cases, performing mass spectrometry comprises identifying at least about 10, 20, 30, 40, 50, 60, 70, 80, 90, 100, or more Chs in the biological sample. In some cases, performing mass spectrometry comprises identifying at least about 1, 2, 3, 4, 5, 6, 7, 8, 9, 10, 20, 30, 40, 50, 60, 70, 80, 90, 100, 500, 1,000, or more diacyl glycerols (DAGs) in the biological sample. In some cases, performing mass spectrometry comprises identifying at least about 10, 20, 30, 40, 50, 60, 70, 80, 90, 100, or more DAGs in the biological sample. In some cases, performing mass spectrometry comprises identifying at least about 1, 2, 3, 4, 5, 6, 7, 8, 9, 10, 20, 30, 40, 50, 60, 70, 80, 90, 100, 500, 1,000, or more lysophosphatidylcholines (LPCs) in the biological sample. In some cases, performing mass spectrometry comprises identifying at least about 10, 20, 30, 40, 50, 60, 70, 80, 90, 100, or more LPCs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1,000, 5,000, 10,000, or more phosphatidylcholines (PCs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more PCs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 500, 1,000, 5,000, 10,000, or more phosphatidylethanolamines (PEs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, or more PEs in the biological sample. In some cases, performing mass spectrometry comprises identifying at least about 1, 2, 3, 4, 5, 6, 7, 8, 9, 10, 20, 30, 40, 50, 60, 70, 80, 90, 100, 500, 1,000, 5,000, 10,000, or more phosphatidylinositols (PIs) in the biological sample. In some cases, performing mass spectrometry comprises identifying at least about 10, 20, 30, 40, 50, 60, 70, 80, 90, 100, or more PIs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 500, 1,000, 5,000, 10,000, or more sphingomyelins (SMs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, or more SMs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1,000, 5,000, 10,000, or more triacylglycerols (TAGs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more TAGs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1,000, 5,000, 10,000, or more cardiolipins (CLs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more CLs in the biological sample. In some cases, performing mass spectrometry comprises identifying at least about 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1,000, 5,000, 10,000, or more free fatty acids (FFAs) in the biological sample. In some cases, performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more FFAs in the biological sample.

In some cases, the panels disclosed herein can be used to identify a number of metabolites, metabolite fragments, metabolite adducts, or metabolite classes using a metabolite analysis workflow. In some cases, metabolite analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). In some cases, metabolite analysis may comprise contacting a sample to distinct surface types (e.g., a particle panel), forming adsorbed biomolecule layers on the distinct surface types to provide an altered composition of the sample (e.g., depleted or enriched for a biomolecule or class of biomolecule, such as proteins or lipids). In order to further isolate specific classes of metabolite, one or more liquid extraction operations (e.g., contacting the altered composition of the biological sample with an organic solvent and/or water) as described herein may be performed to provide an aqueous layer comprising one or more polar metabolites, and the metabolite analysis may comprise identifying one or more polar metabolites in the aqueous layer (e.g., by mass spectrometry). Performing mass spectrometry on a metabolite may produce one or more feature intensities on a plot of mass to charge ratio versus intensity from a mass spectrometry run of a sample comprising the metabolite. In some cases, these features can correspond to variably ionized fragments of metabolites and/or fragments or adducts thereof. In some cases, using the data analysis methods described herein, feature intensities can be sorted into metabolite groups. In some cases, performing mass spectrometry identifies at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000, or more distinct metabolites. In some cases, performing mass spectrometry identifies at most about 100,000, 50,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct metabolites.

In some cases, methods and systems as described herein comprise more than one or all of aspects of proteomic, lipidomic, and metabolomic workflows. Such workflows may generally be referred to as “multiomic” herein. A multiomics workflow as described herein may comprise performing the same or analogous operations on a plurality of classes of biomolecules. For example, a mulitomics workflow may comprise contacting a biological sample or portion thereof with a plurality of surfaces, at least one of which is configured to adsorb a first class of biomolecules (e.g., proteins) and at least another of which is configured to adsorb a second class of biomolecules (e.g., lipids). The multiomics workflow may further comprise identifying and/or quantifying (e.g., by mass spectrometry) at least one protein and at least one lipid adsorbed to the respective surfaces. Alternatively, a multiomics workflow as described herein may comprise performing different sets or subsets of operations on a biological sample to analyze different classes of biomolecules. In an example, a multiomics workflow may comprise an operation of contacting a biological sample or a portion of a biological sample with a surface or plurality of surfaces configured to adsorb one class of biomolecules (e.g., proteins). The multiomics workflow may further comprise a liquid extraction as described herein on the same portion or another portion (e.g., altered composition of) the biological sample. The multiomics workflow may comprise an operation of performing mass spectrometry on the at least one protein and at least one lipid extracted by the liquid extraction (e.g., comprised in an organic layer).

A multiomics workflow as disclosed herein may comprise identifying and/or quantifying (e.g., by mass spectrometry) at least one lipid and at least one metabolite from a biological same. In an example, a multiomics workflow comprises an operation of contacting a biological sample or a portion of a biological sample with a surface or plurality of surfaces configured to adsorb one class of biomolecules (e.g., lipids). The multiomics workflow may further comprise a liquid extraction as described herein on the same portion or another portion (e.g., altered composition of) the biological sample. The multiomics workflow may comprise an operation of performing mass spectrometry on the at least one lipid and at least one lipid extracted by the liquid extraction (e.g., comprised in an aqueous layer). An organic layer form the liquid extraction may additionally be subjected to identification and/or quantifications.

A multiomics workflow as described herein may comprise identifying at least one each of a protein, a lipid, and a metabolite. In an example, a multiomics workflow may comprise an operation of contacting a biological sample or a portion of a biological sample with a surface or plurality of surfaces configured to adsorb one class of biomolecules (e.g., proteins). The multiomics workflow may further comprise a liquid extraction as described herein on the same portion or another portion (e.g., altered composition of) the biological sample. The multiomics workflow may comprise an operation of performing mass spectrometry on the at least one protein, at least one lipid extracted by the liquid extraction (e.g., comprised in an organic layer), and at least one metabolite extracted by the liquid extraction (e.g., comprised in an aqueous layer).

### Biological Sample

A biological sample can comprise a single sample or a plurality of samples from a species, an individual organism, or a part of an individual organism. In some cases, the biological sample can be obtained from an individual organism. In some cases, the biological sample can comprise a plurality of samples obtained from a population of organisms. In some cases, the biological sample can comprise a gene. In some cases, the biological sample can comprise a tissue. In some cases, the biological sample can comprise an organ. In some cases, the biological sample can be obtained by performing a biopsy. In some cases, the biological sample can be obtained by performing a tissue biopsy. In some cases, the biological sample can comprise a tumor biopsy. In some cases, the biological sample can comprise a liquid biopsy. In some cases, the biological sample may be processed (e.g., lysed, blended, centrifuged, fractionated, etc.). In some cases, the biological sample may comprise media comprising biomolecules secreted by one or more cells. In some cases, the biological sample may be cell-free or substantially cell-free. In some cases, the biological sample may comprise a plurality of biomolecules. In some cases, a plurality of biomolecules may comprise lipids. In some cases, a plurality of biomolecules may comprise metabolites. In some cases, a plurality of biomolecules may comprise proteins. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic or function (e.g., proteins, nucleic acids, carbohydrates, lipids, metabolites).

In some cases, the biological sample disclosed herein comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, the biological sample comprises blood, serum, or plasma, or any portion or fraction thereof. In some cases, the biological sample comprises blood. In some cases, the biological sample comprises blood or any portion or fraction thereof. In some cases, the blood is diluted. In some cases, the biological sample comprises serum. In some cases, the biological sample comprises serum or any portion or fraction thereof. In some cases, the serum is diluted. In some cases, the biological sample comprises plasma. In some cases, the biological sample comprises plasma or any portion or fraction thereof. In some cases, the plasma is diluted.

In some cases, the biological sample can comprise a cell. In some cases, a cell can refer to a basic unit of life comprising at least a cellular membrane and genetic material. In some cases, a biological sample can comprise a cell of a single-celled organism. In some cases, a biological sample can comprise a cell of a multicellular organism. In some cases, a biological sample can comprise a bacterial cell. In some cases, a biological sample can comprise a fungal cell. In some cases, a biological sample can comprise a virus-infected cell. In some cases, a biological sample can comprise a mammalian cell. In some cases, a biological sample can comprise a human cell. In some cases, a biological sample can comprise a specialized cell in a multicellular organism. In some cases, a biological sample can comprise a stem cell. In some cases, a biological sample can comprise a healthy cell. In some cases, a biological sample can comprise a cancerous cell. In some cases, a biological sample can comprise a malignant cell. In some cases, a biological sample may comprise lipids and various forms thereof. In some cases, a biological sample may comprise metabolites and various forms thereof. In some cases, a biological sample may comprise nucleic acids and various forms thereof. In some cases, a biological sample may comprise proteins and various forms thereof. In some cases, a cell is part of a plurality of cells. In some cases, the plurality of cells are cells of a same type. In some cases, the cell is of a tissue sample, an organoid, an immortalized cell line, or any combination thereof. In some cases, the cell is a stem cell. In some cases, the cell is afflicted with an infection or a mutation. In some cases, the cell is a viable cell comprising a cancer cell, an epithelial cell, a bone cell, a muscle cell, a fat cell, a tissue cell, or nerve cell. In some cases, the cancer cell is a biopsied cell of a patient. In some cases, the cell is a eukaryote or a prokaryote. In some cases, the biological sample can comprise a yeast. In some cases. In some cases, the plurality of cells is comprised in a tissue, an organoid, an organism, or a plurality of organisms. In some cases, the cell is derived from an immortalized cell line. In some cases, the cell is a HeLa cell. In some cases, the cell is a stem cell. In some cases, the cell is comprised in a primary cell culture. In some cases, the cell comprises a genetically modified cell.

A subject can comprise any living organism. In some cases, a subject can be a cell. In some cases, a subject can comprise a bacterium, a mammalian cell, a human cell, a fungal cell, a colony of bacteria, a tissue of a mammal, an organ of a mammal, a mammal, a tissue of a human, an organ of a human, a fungus, or any combination thereof. In some cases, a subject can comprise a cancer cell, a healthy cell, or both.

In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000, or more distinct proteins. In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct proteins. In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000, or more distinct protein groups. In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct protein groups.

In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 50,000, 100,000, or more distinct lipids or metabolites. In some cases, the biological sample comprises at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids or metabolites.

In some cases, the biological sample comprises no more than about 100,000, 50,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites. In some cases, the biological sample comprises no more than about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites.

Lipids comprised in biological samples as disclosed herein may comprise one or more of a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a sphingomyelin (SM), a free fatty acid (FFA), or a cardiolipin (CL), or any derivative thereof.

Metabolites comprised in biological samples as disclosed herein may comprise one or more of a vitamin, a cofactor, a nucleotide, a polynucleotide, an amino acid or analogue thereof, a peptidomimetic, an organic acid, an alcohol, a diol, a polyol, a monosaccharide, a disaccharide, an oligosaccharide, a polysaccharide, a hydrocarbon, a benzenoid, an alkaloid, an acetylide, a polyketide, a terpene or terpenoid, a phenolic, or any combination or derivative thereof.

### Apparatus

In some aspects, the present disclosure describes an apparatus for assaying a biological sample. Apparatuses may be configured to carry out any of the methods or operations disclosed herein.

### Automated Sample Preparation

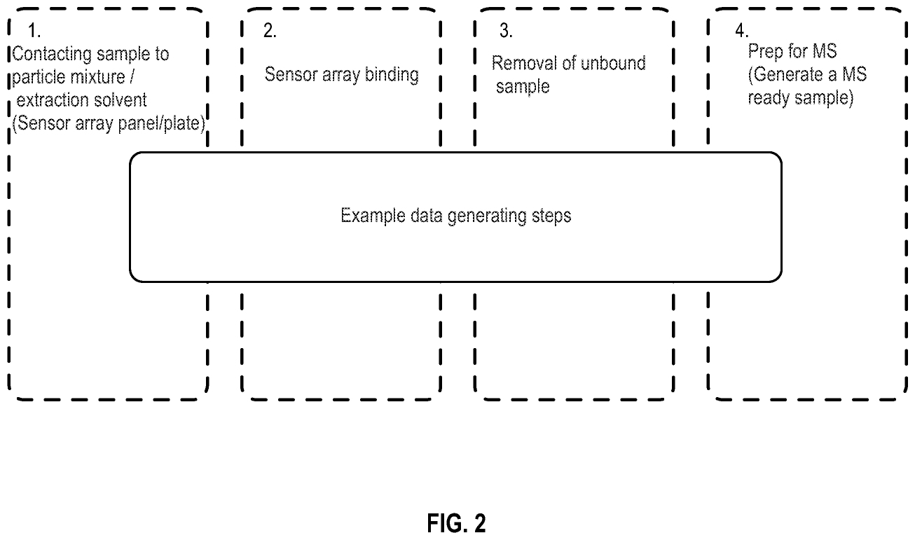

The present disclosure provides systems and methods for automated sample preparation, data generation, and biological sample analysis. These systems and methods may prepare samples for analysis using the mass spectrometry-based analysis methods disclosed herein. As is depicted in FIG. 2, the systems and methods can comprise (1) contacting a sample to surfaces (e.g., to particles in a particle mixture) on a sensor array, substrate, plate, or within partitions on any of the foregoing and/or with a solvent as part of a liquid extraction operation as described herein; (2) allowing biomolecules in the sample to bind to the surfaces; (3) removing unbound sample from the surfaces, and (4) preparing a sample for analysis (e.g., using mass spectrometry (“MS”)). For example, in (1), a method of the present disclosure can comprise contacting a biological sample to a plurality of particles. Alternatively, or additionally, (1) can comprise contacting the biological sample with a solvent system as described herein. In (2), the sample may be incubated with the plurality of particles so as to promote biomolecule adsorption to the particles. In (3), unbound sample may be removed while retaining the particles and the biomolecules adsorbed to the particles. In (4) the adsorbed biomolecules may be desorbed from the particles and preparing them for mass spectrometric analysis by which example data may be generated. Example of suitable automated sample methods and systems include, but are not limited to, those disclosed in U.S. Publication No. 2021/0285957, which is hereby incorporated by reference in its entirety.

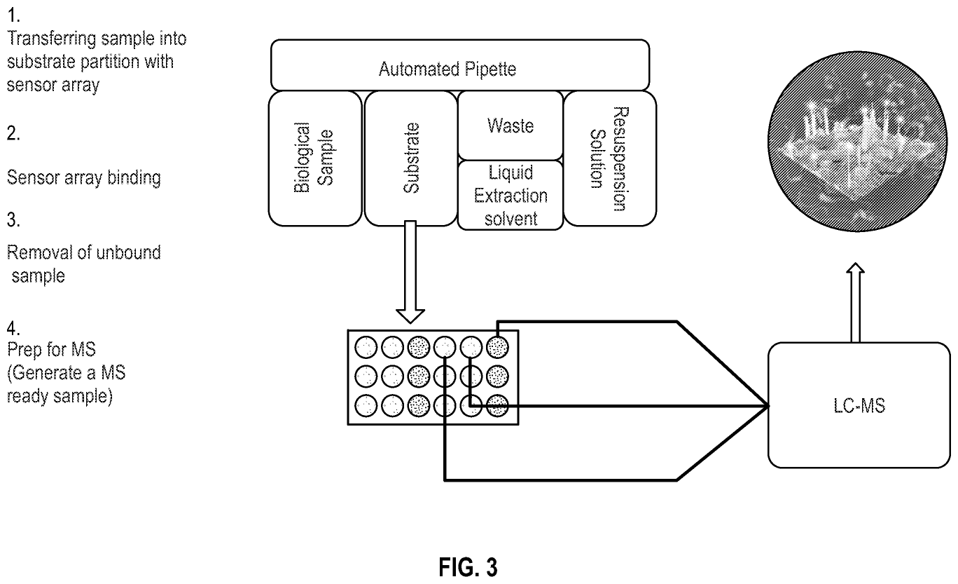

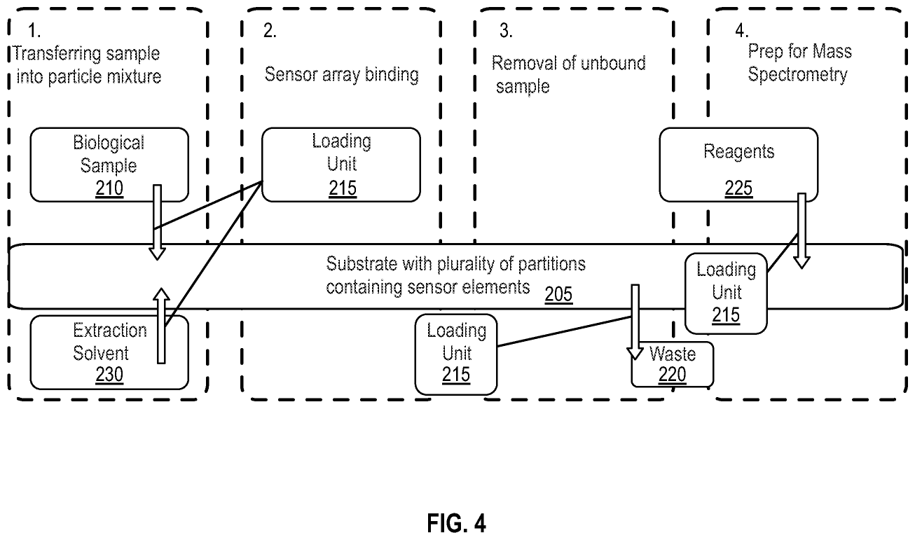

The present disclosure provides automated systems, methods and kits for biomolecule corona preparation and analysis. The automated apparatus may perform at least the aforementioned data generating steps outlined in FIG. 2 using various units illustrated in FIG. 3 and FIG. 4. The automated apparatus may contain a substrate with a plurality of partitions containing sensor elements 205 and a biological sample 210. The loading unit 215 on the apparatus may transfer a portion of the biological sample 210 into a partition on the substrate 205, leading to adsorption of biomolecules from the biological sample onto a sensor element in the partition on the substrate. The automated apparatus may then remove unbound biomolecules from the partition, optionally transferring the unbound sample into a waste receptacle 220. Alternatively, or additionally, the loading unit 215 may transfer one or more extraction solvents 230 as described herein to the sample, optionally in contact with one or more surfaces. The remaining biomolecules (e.g., biomolecules adsorbed to the sensor element or comprised in an organic layer) may be desorbed, collected, and prepared for mass spectrometric analysis. The reagents 225 may comprise a buffer, such as a resuspension buffer capable of desorbing biomolecules from a biomolecule corona or a denaturation buffer capable of denaturing or fragmenting a biomolecule. Reagents (e.g., a buffer or protease) 225 may also be loaded using the loading unit 215 to facilitate any of the foregoing.

In some aspects, the present disclosure provides an automated system comprising a network of units with differentiated functions in distinguishing states of a complex biological sample using a plurality of particles having surfaces with different physicochemical properties wherein: a first unit comprises a multichannel fluid transfer instrument for transferring fluids between units within the system; a second unit comprises a support for storing a plurality of biological samples; a third unit comprises a support for a sensor array plate (e.g., a substrate comprising a plurality of partitions comprising sensor elements, such as a 96 well plate containing nanoparticles) possessing partitions that comprise the plurality of particles having surfaces with different physicochemical properties for detecting a binding interaction between a population of analytes within the complex biological sample and the plurality of particles; a fourth unit comprises supports for storing a plurality of reagents; a fifth unit comprises supports for storing a reagent to be disposed of; a sixth unit comprises supports for storing consumables used by the multichannel fluid transfer instrument; and wherein the system is programed to perform a series of steps comprising: contacting the biological sample with a specified partition of the sensor array; incubating the biological sample with the plurality of particles contained within the partition of the sensor array plate; removing all components from a partition except the plurality of particles and a population of analytes interacting with a particle; and preparing a sample for mass spectrometry.

An example of such an apparatus is provided in FIG. 4. The apparatus comprises an automated pipette that is able to transfer volumes between a biological sample storage unit, a substrate comprising a plurality of partitions comprising a plurality of sensor elements, a waste collection unit, a unit comprising a denaturation solution, and a unit comprising a resuspension solution. The automated apparatus can perform a biomolecule corona assay which comprises transferring a portion of the biological sample into a partition within the substrate comprising a sensor element, incubating the portion of the sample with the sensor element to allow biomolecules from the biological sample to bind to the sensor element, removing contents from the partition comprising biomolecules that are not bound to the sensor elements, and then preparing the biomolecules that remained within the partition for mass spectrometric (MS) analysis (e.g., LC-MS).

The loading may comprise a degree of mobility that enables access to all other unit within the system. The loading may comprise a capacity to perform pipetting functions.

The system or apparatus of the present disclosure may comprise support for a single plate, a 6 well plate, a 12 well plate, a 96 well plate, a 192 well plate, a 384 well plate, or a rack of microtubes. In some embodiments, the system or apparatus of the present disclosure may comprise a thermal unit capable of modulating the temperature of said support and a sample. In some embodiments, the system or apparatus of the present disclosure may comprise a rotational unit capable of physically agitating and/or mixing a sample.

In some embodiments, the plurality of particles comprises surfaces with different physicochemical properties for detecting a binding interaction between a population of analytes within the complex biological sample and the plurality of particles are immobilized to a surface with a partition of the sensory array. In some embodiments, the plurality of particles comprises a plurality of magnetic nanoparticles in a solution with different physicochemical properties for binding a population of analytes within the complex biological sample and the plurality of particles. In some embodiments, the system comprises a step wherein the sensor array plate is transferred to an additional seventh unit that comprises a magnetized support and a thermal unit capable of modulating the temperature of said support and a sample and incubated for an additional amount of time.

In some embodiments, the fourth unit comprises a set of reagents for: generating the sensor array plate; washing an unbound sample; and/or preparing a sample for mass spectrometry. In some embodiments, contacting the biological sample with a specified partition of the sensor array comprises pipetting a specified volume of the biological sample into the specific partition of the sensor array. In some embodiments, contacting the biological sample with a specified partition of the sensor array comprises pipetting a volume corresponding to a 1:1, 1:2:1:3, 1:4, 1:5, 1:6, 1:7, 1:8, 1:9, 1:10, 1:15, or 1:20 ratio of a plurality of particles in a solution to the biological sample.

In some embodiments, contacting the biological sample with a specified partition of the sensor array comprises pipetting a volume of at least 10 microliters, at least 50 microliters, at least 100 microliters, at least 250 microliters, at least 500 microliters, or at least 1000 microliters the biological sample into the specific partition of the sensor array

In some cases, the apparatus comprises a computer-readable medium. In some cases, the computer-readable medium comprises machine-executable code. In some cases, the machine-executable code, upon execution by one or more processors programmed individually or collectively, implements a method comprising: (i) transferring at least a portion of the biological sample to the surface and (ii) adding a first solvent to at least a portion of the altered composition of the biological sample.

### Kits

In some aspects, the present disclosure describes a kit for assaying a biological sample. In some cases, the kit comprises a substrate. In some cases, the substrate comprises a surface (e.g., a particle, as described herein). In some cases, the substrate comprises a surface configured to bind a biomolecule in the biological sample. In some cases, the kit comprises a first organic solvent. A kit may comprise instructions for using the substate and the solvent to process the sample to adsorbed (e.g., on a surface of the substrate) one or more biomolecules. The kit may comprise instructions for using the solvent to process the sample to produce an altered composition of the biological sample. The altered composition of the biological sample may comprise an organic layer comprising a lipid form the biological sample. The altered composition of the biological sample may comprise an aqueous layer comprising a polar metabolite form the biological sample. The kit may further comprise instructions for preparing the sample, altered composition of the sample, or a portion or fraction thereof for downstream analysis to identify one or more biomolecules.

In some cases, the instructions comprise instructions for performing any method disclosed herein. In some cases, the instructions comprise instructions for using any system as disclosed herein.

### Computer Systems

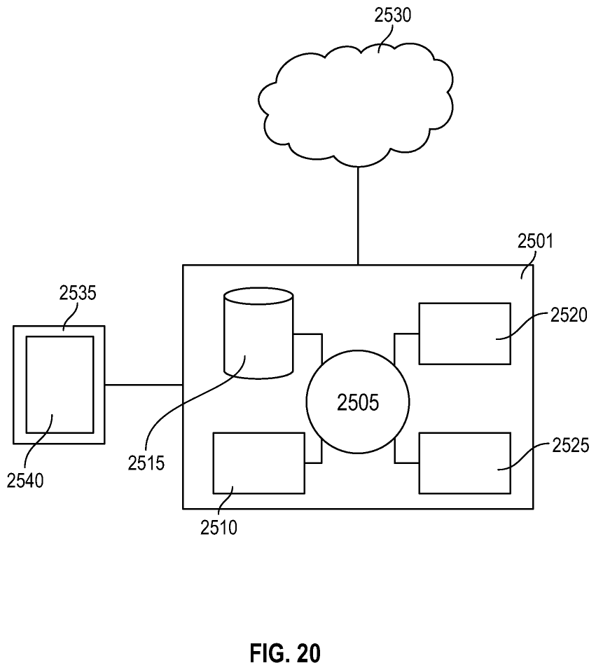

The present disclosure provides computer systems that are programmed to implement methods of the disclosure or to control systems of the present disclosure. FIG. 20 shows a computer system 2501 that is programmed or otherwise configured to, for example, culture a cell, transport biological samples, perform an assay for biomolecules, run analytical instruments, analyze mass spectra, or any combination thereof.

The computer system 2501 may regulate various aspects of analysis, calculation, and generation of the present disclosure, such as, for example, for culturing a cell, transporting biological samples, performing an assay for biomolecules, running analytical instruments, analyzing mass spectra or any combination thereof. The computer system 2501 may be an electronic device of a user or a computer system that is remotely located with respect to the electronic device. The electronic device may be a mobile electronic device.

The computer system 2501 includes a central processing unit (CPU, also “processor” and “computer processor” herein) 2505, which may be a single core or multi core processor, or a plurality of processors for parallel processing. The computer system 2501 also includes memory or memory location 2510 (e.g., random-access memory, read-only memory, flash memory), electronic storage unit 2515 (e.g., hard disk), communication interface 2520 (e.g., network adapter) for communicating with one or more other systems, and peripheral devices 2525, such as cache, other memory, data storage and/or electronic display adapters. The memory 2510, storage unit 2515, interface 2520 and peripheral devices 2525 are in communication with the CPU 2505 through a communication bus (solid lines), such as a motherboard. The storage unit 2515 may be a data storage unit (or data repository) for storing data. The computer system 2501 may be operatively coupled to a computer network (“network”) 2530 with the aid of the communication interface 2520. The network 2530 may be the Internet, an internet and/or extranet, or an intranet and/or extranet that is in communication with the Internet.

The network 2530 in some cases is a telecommunication and/or data network. The network 2530 may include one or more computer servers, which may enable distributed computing, such as cloud computing. For example, one or more computer servers may enable cloud computing over the network 2530 (“the cloud”) to perform various aspects of analysis, calculation, and generation of the present disclosure, such as, for example, culturing a cell, transporting biological samples, performing an assay for biomolecules, running analytical instruments, analyzing mass spectra, or any combination thereof. Such cloud computing may be provided by cloud computing platforms such as, for example, Amazon Web Services (AWS), Microsoft Azure, Google Cloud Platform, and IBM cloud. The network 2530, in some cases with the aid of the computer system 2501, may implement a peer-to-peer network, which may enable devices coupled to the computer system 2501 to behave as a client or a server.

The CPU 2505 may comprise one or more computer processors and/or one or more graphics processing units (GPUs). The CPU 2505 may execute a sequence of machine-readable instructions, which may be embodied in a program or software. The instructions may be stored in a memory location, such as the memory 2510. The instructions may be directed to the CPU 2505, which may subsequently program or otherwise configure the CPU 2505 to implement methods of the present disclosure. Examples of operations performed by the CPU 2505 may include fetch, decode, execute, and writeback.

The CPU 2505 may be part of a circuit, such as an integrated circuit. One or more other components of the system 2501 may be included in the circuit. In some cases, the circuit is an application specific integrated circuit (ASIC).

The storage unit 2515 may store files, such as drivers, libraries and saved programs. The storage unit 2515 may store user data, e.g., user preferences and user programs. The computer system 2501 in some cases may include one or more additional data storage units that are external to the computer system 2501, such as located on a remote server that is in communication with the computer system 2501 through an intranet or the Internet.

The computer system 2501 may communicate with one or more remote computer systems through the network 2530. For instance, the computer system 2501 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user may access the computer system 2501 via the network 2530.

Methods as described herein may be implemented by way of machine (e.g., computer processor) executable code stored on an electronic storage location of the computer system 2501, such as, for example, on the memory 2510 or electronic storage unit 2515. The machine executable or machine-readable code may be provided in the form of software. During use, the code may be executed by the processor 2505. In some cases, the code may be retrieved from the storage unit 2515 and stored on the memory 2510 for ready access by the processor 2505. In some situations, the electronic storage unit 2515 may be precluded, and machine-executable instructions are stored on memory 2510.

The code may be pre-compiled and configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 2501, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2501 may include or be in communication with an electronic display 2535 that comprises a user interface (UI) 2540 for, for example, culturing a cell, transporting biological samples, performing an assay for biomolecules, running analytical instruments, analyzing mass spectra, or any combination thereof. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 2505. The algorithm can, for example, culture a cell, transport biological samples, perform an assay for biomolecules, run analytical instruments, analyze mass spectra, of any combination thereof.

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present cases disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one,” “one or more,” and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and,” “or,” or “and/or” and may be used both exclusively and inclusively. For example, the term “A or B” may refer to “A or B,” “A but not B,” “B but not A,” and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred cases of the present invention have been shown and described herein, it will be obvious to those skilled in the art that such cases are provided by way of example only. It is not intended that the invention be limited by the specific examples provided within the specification. While the invention has been described with reference to the aforementioned specification, the descriptions and illustrations of the cases herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the cases of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following examples are provided to further illustrate some cases of the present disclosure but are not intended to limit the scope of the disclosure; it will be understood by their exemplary nature that other procedures, methodologies, or techniques known to those skilled in the art may alternatively be used.

### Example 1: Sample Preparation Workflow

As shown in FIG. 1D, the general workflow for lipid extractions may involve adding one or more organic solvents and water to a biological sample, centrifuging the mixture, allowing the mixture to separate into multiple phases, which may include but are not limited to an organic phase comprising lipids, an aqueous phase comprising polar metabolites, and a protein pellet. Each of these layers may be dried down and analyzed using mass spectrometry as described herein. In some cases, the organic layer may be analyzed by reverse phase liquid chromatograph (RPLC)-MS. In some cases, the aqueous layer may be analyzed by hydrophilic interaction chromatograph (HILIC)-MS.

### Example 2: Sample Preparation by the Folch Extraction Method

The Folch method provides a method for extracting lipids in a sample using a biphasic mixture of chloroform or dichloromethane and methanol.

In an exemplary embodiment, about 40-200 μL of plasma is added into about 1-2 mL of dichloromethane (DCM):methanol (MeOH) at about a 1:1 ratio (v/v). About 120 μL of water is added to the mixture. Then, the mixture is centrifuged for about 10 min, resulting in two layers. The bottom layer (i.e., DCM with lipids) is extracted. Optionally, the bottom layer may be extracted with another aliquot of DCM. The extracted bottom layer was dried down. The dried-down bottom layer may be reconstituted for MS analysis as provided herein.

In some cases, the Folch method may be difficult to integrate into an automated workflow. In some cases, the Folch method may be incompatible with plastic, such as polypropylene (PP), or materials that are commonly used in automated liquid handling. In some cases, the chloroform or DCM layer being on the bottom of the phase separation may be challenging for an automated liquid handling apparatus to collect. Good ventilation may be needed to due to potential for inhalation toxicity with the solvents used in this method. In some cases, the potential for fast evaporation of the chloroform or DCM layer may be advantageous in quickly drying the sample down.

### Example 3: Sample Preparation by Extraction Using Methyl Tert-Butyl Ether (MTBE)

MTBE extraction provides a method for extracting lipids in a sample using MTBE. In an exemplary embodiment, about 40 μL of plasma is added into about 260 μL of MeOH. Up to about 1 mL of MTBE is added to the mixture and mixed for about 10 min. About 250 μL of water is added into the mixture. Then, the mixture is centrifuged for about 10 min, resulting in two layers. The upper layer containing MTBE and lipids is taken. If desired, the upper layer may be extracted with another aliquot of MTBE. The extracted bottom layer is dried down. The dried-down layer may be reconstituted for MS analysis as provided herein.

In some cases, the MTBE method may perform similarly to the Folch method using chloroform in extracting lipids from a sample. In some cases, the MTBE method may be compatible with plastic, such as polypropylene (PP), or materials that are commonly used in automated liquid handling. Good ventilation may be needed to due to potential for inhalation toxicity with the solvents used in this method. In some cases, the high vapor pressure of MTBE may pose issues with evaporation and sealing of the sample plate.

### Example 4: Sample Preparation by Butanol/Methanol (BUME) Biphasic Extraction Method

BUME extraction provides a method for extracting lipids in a sample using a butanol:methanol mixture. In an exemplary embodiment, about 10-100 μL of plasma is added into 300 μL of butanol:MeOH (1:1 v/v), and the mixture was mixed for 10 min. Into the mixture, 150 μL of heptane:ethyl acetate (3:1 v/v) was added and mixed for 5 min. Then, 300 μL of water was added into the mixture and mixed for 5 min. After waiting for 5 min for separation, the upper layer (i.e., organic solvent mixture with lipids) was removed and dried down. The dried-down layer may be reconstituted for MS analysis as provided herein.

The BUME method was designed to be more compatible with an automated workflow. In some cases, the BUME method may have a better recovery of polar lipids than MTBE or DCM extraction. In some cases, heptane and ethyl acetate used in the BUME method may not be compatible with plastic, such as polypropylene (PP), or materials that are commonly used in automated liquid handling. A good ventilation may be needed to due to potential for inhalation toxicity with the solvents used in this method. In some cases, solvents used in the BUME method have lower vapor pressure than MTBE and may avoid issues with evaporation and sealing of the sample plate. In some cases, the phase formation may occur spontaneously by BUME method.

### Example 5: Sample Preparation by Butanol/Methanol Single Extraction Method

The butanol/methanol single phase extraction provides a variation on the biphasic BUME method for extracting lipids in a sample. In an exemplary embodiment, 10 μL of plasma was added into 100 μL of butanol:MeOH and mixed for 10 min. After the mixtures was centrifuged for 10 min, a supernatant was taken and injected directly into mass spectrometry or dried down. If desired, a second extraction may be performed before MS analysis as provided herein.

The butanol/methanol single phase extraction method provides a simple workflow that may be compatible with plastic, such as polypropylene (PP), or materials that are commonly used in automated liquid handling. In some cases, the butanol/methanol single phase extraction method does not require a phase separation, which simplifies the workflow compared to other approaches. In some cases, the potential for inhalation toxicity with the solvents used in this method may be lower than with other solvents used for lipid extraction.

### Example 6: Methods for Lipidomics Mass Spectrometry

Representative conditions for lipidomics mass spectrometry may use the following conditions and settings:
Column: Kinetex C18;
Mobile phase A: 60:40 acetonitrile:water with 10 mM ammonium acetate;
Mobile phase B: 90:10 isopropanol:acetonitrile with 10 mM ammonium acetate;
Flow rate: 0.25 mL/min; and
Gradient: 15% B to 100% B over 30 min with 5 min of equilibration. Due to lipids isomers, run length time may be extended for better separation.
Data was processed with MS-Dial for peak integration and identification.

### Example 7: Methods for Metabolomics Mass Spectrometry

Representative conditions for metabolomics mass spectrometry may use the following conditions and settings:
Instrument: Sciex 6600 QToF in positive and negative mode
HILIC:
   Column: ZIC-HILIC
   Flow rate: 0.5 mL/min
   Mobile phase A: 50:50 water:acetonitrile with 10 mM ammonium acetate
   Mobile phase B: 95:5 acetonitrile:water with 10 mM ammonium acetate
   Gradient: 100% B to 40% B over 25 minutes with 5 minutes of equilibration
RPLC:
   Column: Kinetex C18
   Flow rate: 0.25 mL/min
   Mobile phase A: Water with 0.1% Formic Acid
   Mobile phase B: Methanol with 0.1% Formic Acid
   Gradient: 0% B to 100% B over 20 minutes with 5 minutes of equilibration
Data was processed with XCMS, and metabolites were identified with MetID.

### Example 8: Metabolomics and Lipidomics Profiling Using Nanoparticle Coronas

This example described metabolomics and lipidomics profiling using nanoparticle coronas, in accordance with some embodiments of the disclosure.

Functionalized nanoparticles (5 types) were incubated with 40 μL of plasma at 37° C. for 1 h. The nanoparticles were washed twice with buffer then twice with water, and methanol was added.

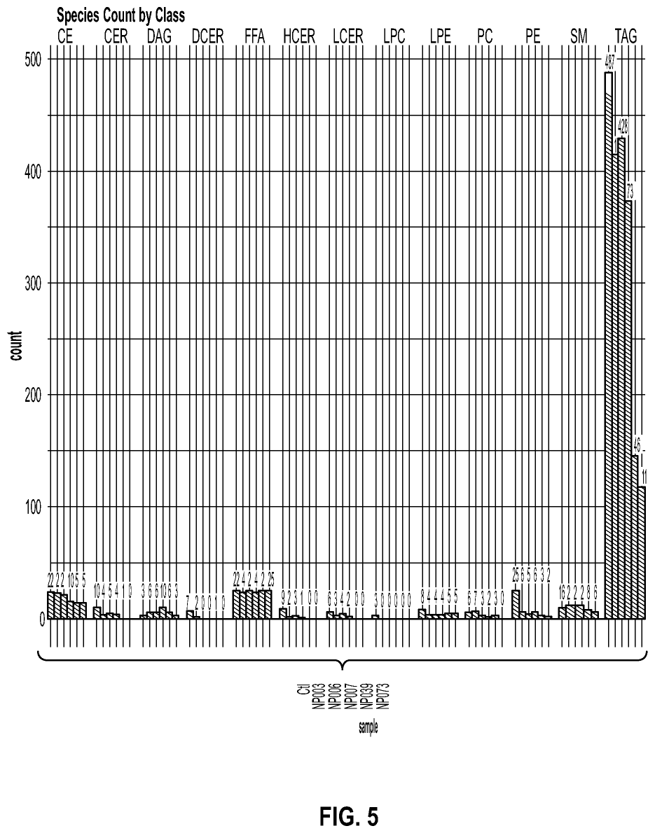

Lipidomics targeted MS/MS (MS2) runs included Lipiyzer™ MRM transitions on Sciex 6500+ and gradients run on a Kinetex C18 column. Species counts per nanoparticle type and neat plasma control (Ctl) are shown in FIG. 5. 2807 lipids in total were identified across nanoparticles. Without being bound to particular theory, nanoparticles may be attaching chylomicrons due to high triacylglycerols (TAG), phosphatidylcholine (PC), and cholesterol ester (CE) species counts and intensities. Sphingomyelins (SMs) are also present in most particles and the control. Other classes (CER: ceramides; DAG: diacylglycerols, DCER: dihydroceramide, FFA: free fatty acids; HCER: hexosylceramide, LCER: lactosylceramide, LPC: lysophosphatidylcholines, LPE: lysophosphatidylethanolamines, PC: phosphatidylcholines, PE: phosphatidylethanolamines) were not detected by the nanoparticles or showed a noisy signal.

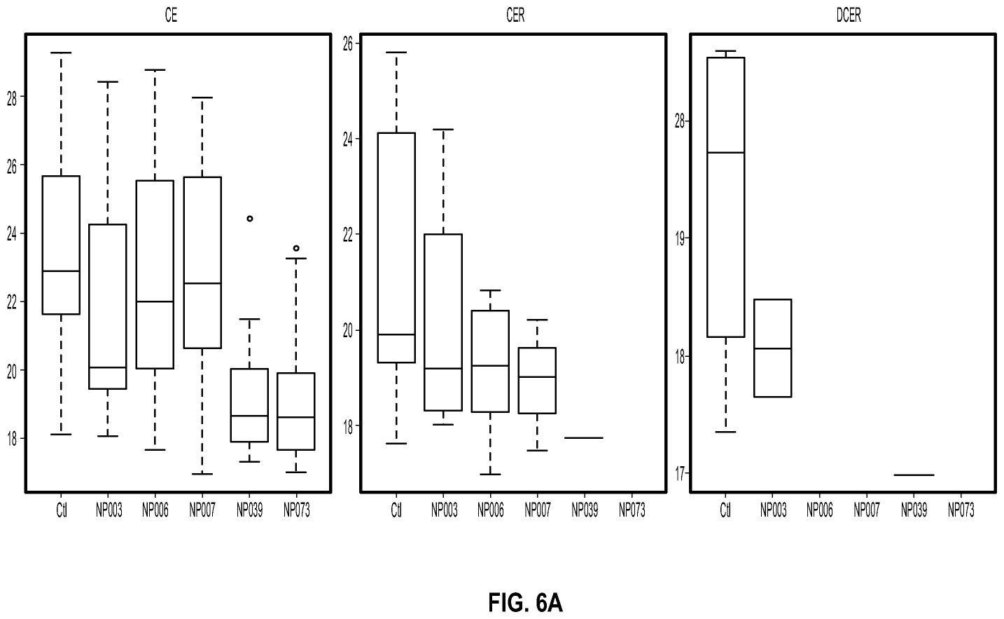

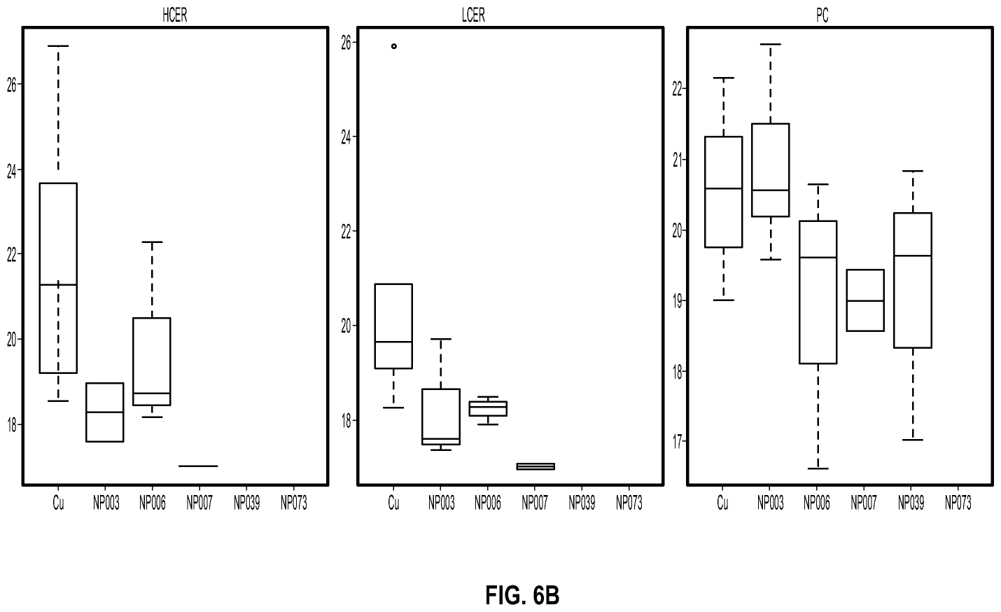

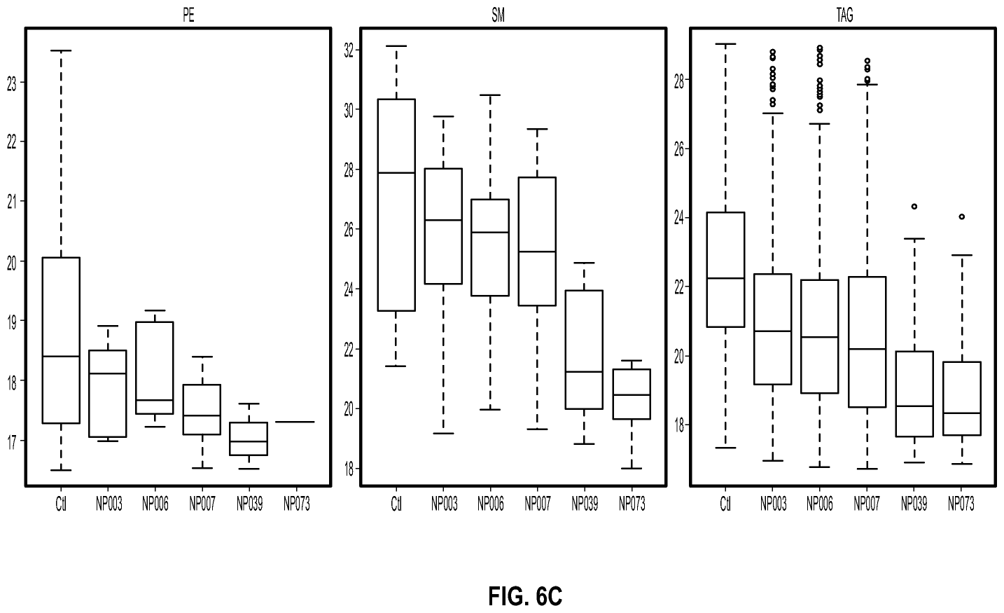

Additional intensity plots of individual classes of lipids across nanoparticles are further illustrated in FIGS. 6A-6C

In an untargeted (MS1) experiment, “Supernatant” (-S) samples were created by adding methanol to plasma samples, crashing out proteins, centrifuging, and then taking the supernatant. The supernatant was incubated with nanoparticles. An “All” (-A) sample was created by incubating nanoparticles in methanol with plasma. Nanoparticles were incubated with the precipitated proteins. All samples were processed in positive and negative mode in Sciex 660QTOF using a Phenomenex C18 column. Data was processed using MS-Dial. Control prep samples (using no nanoparticles) are labeled “Prep.”

335 lipids were annotated between positive and negative mode. The results were Lipids where NP intensity/Blank Intensity>1.5 and NP Intensity/Control Intensity>1:
NP 003:52: by MS2, 472 by MS1
NP006: 57 by MS2, 552 by MS1
NP007: 58 by MS2, 563 by MS1
NP039: 53 by MS2, 541 by MS1
NP073: 51 by MS2, 507 by MS1.

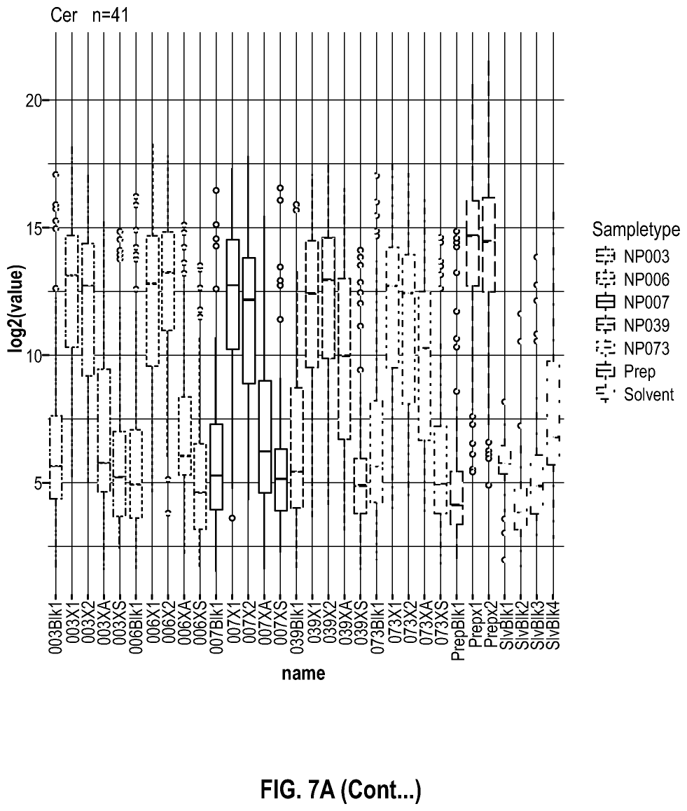

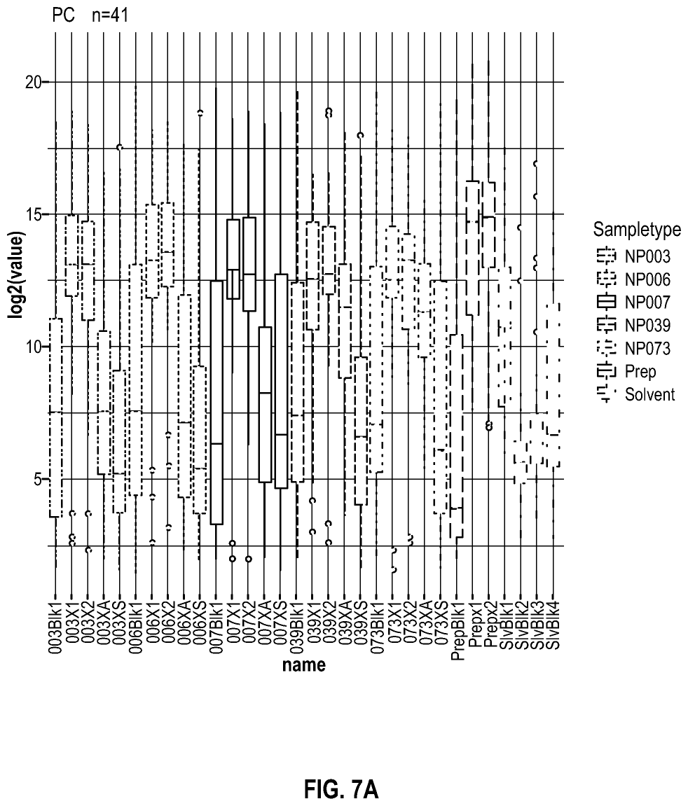

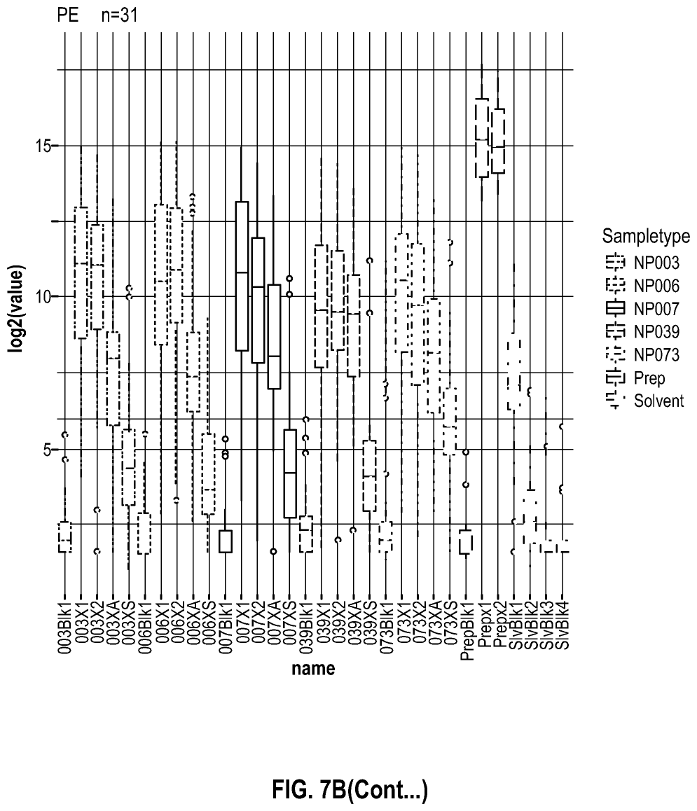

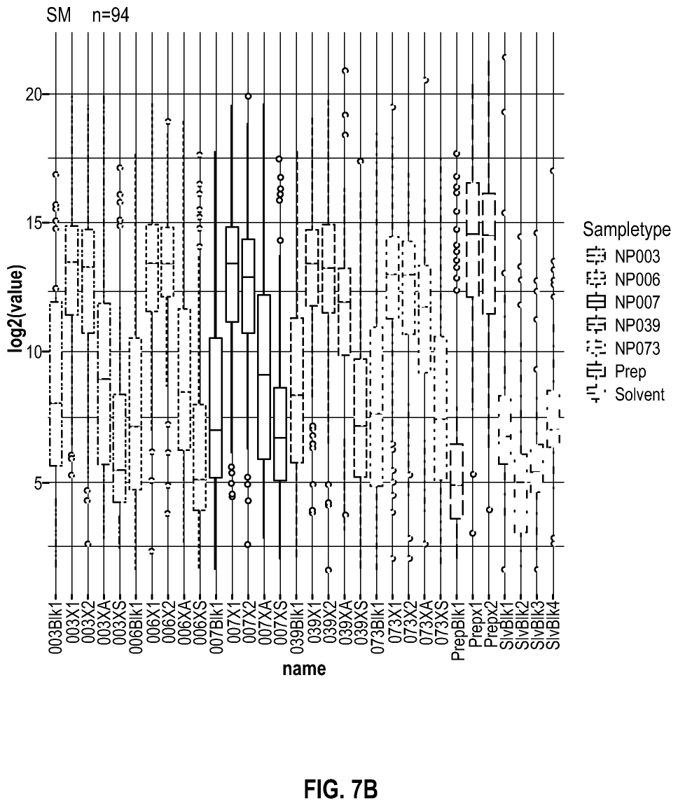

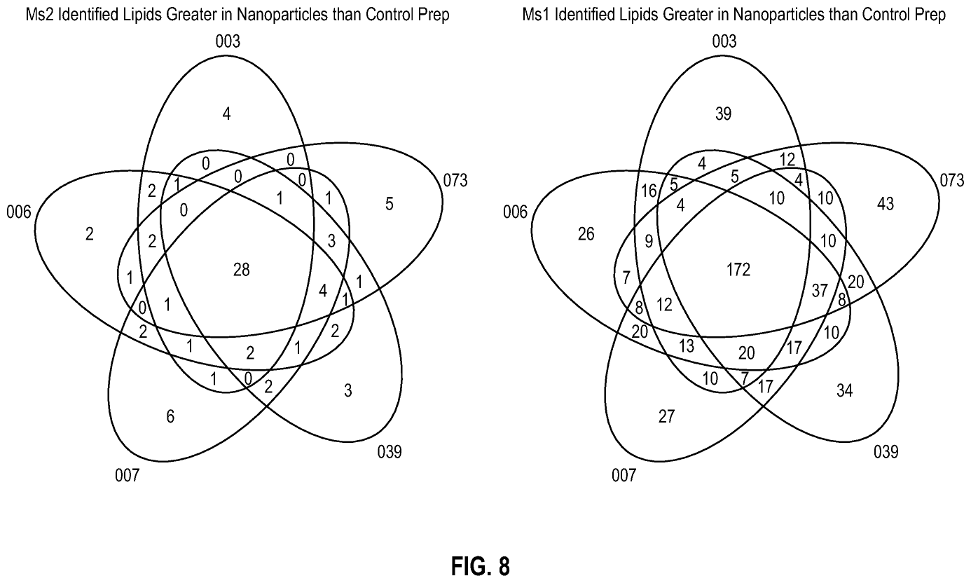

Box plots illustrating the observed intensities of CERs, PCs, PEs, and SMs across particle type are shown in FIGS. 7A and 7B. Venn diagrams of the total number of lipids with intensity greater than control by particle type are shown in FIG. 8 (Left panel: MS2; Right panel: MS1).

### Example 9: Lipid Extraction with Butyl Acetate and Multiomic Analysis

Two different sample preparation methods for lipid extraction as provided herein were compared for types and amount of lipids extracted as well as reproducibility and variability across trials. Samples were also subjected to metabolomics analysis to assess any differences in the type or amounts of metabolites extracted as well as differences in variability across samples using each solvent.

Human plasma samples were subjected to liquid-liquid extraction with either butyl acetate (BA) or methyl tert-butyl ether (MTBE), per the following protocol. Briefly, 40 μL of SPLASH® LIPIDOMIX® and 40 L of a metabolite standard mix were added 50 μL of human plasma 200 μL of a butanol:methanol mixture (3:1 v/v) was added to the spiked-in sample and spun down for 30 min. 500 μL of organic solvent (BA or MTBE) and 300 μL of water added and the extraction mixture was centrifuged for 10 min at 10,000 g. In each case, 500 μL of the organic layer (BA or MTBE and butanol:methanol) was transferred to a new tube and dried down for mass spectrometry analysis of lipid molecules. The 200 μL of the organic layer and the water layer were separately spiked-in with another metabolite standard mix and subjected to mass spectrometry analysis to detect polar metabolites. Mass spectrometry analysis was performed as described in Example 6 and Example 7 above.

While methyl tert-butyl ether (MTBE) may be useful in metabolomics or lipidomics workflows for its ability extract a wide range of lipids, MTBE may not be suitable for at least some (e.g., automated) workflows due to its toxicity, volatility, low viscosity, and low cohesion. Butyl acetate (BA) may avoid some or all of these drawbacks while showing comparable identification results to samples processed using MTBE across a plurality of replicates.

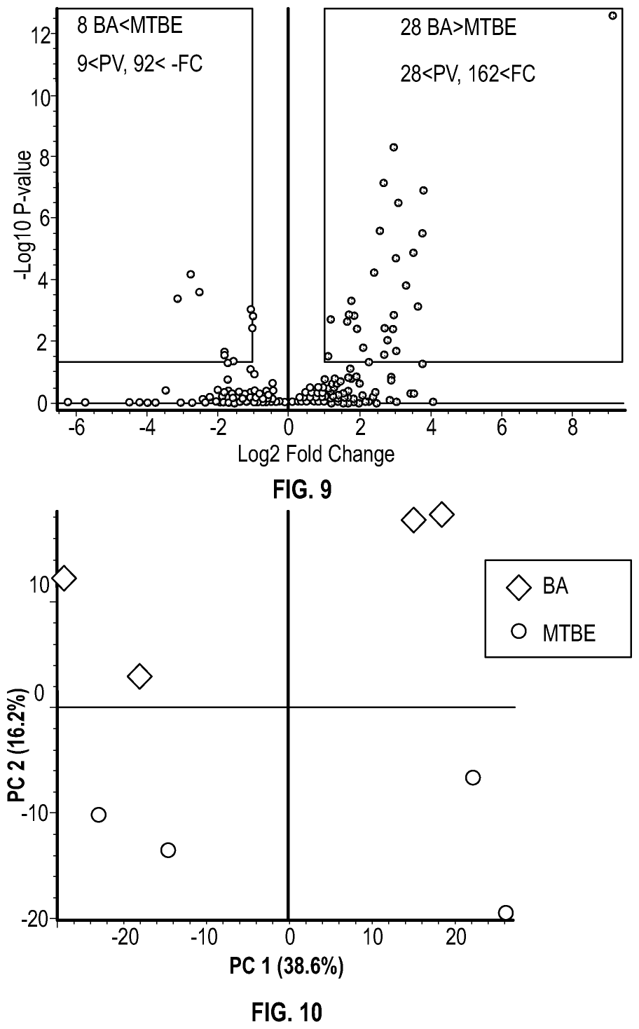

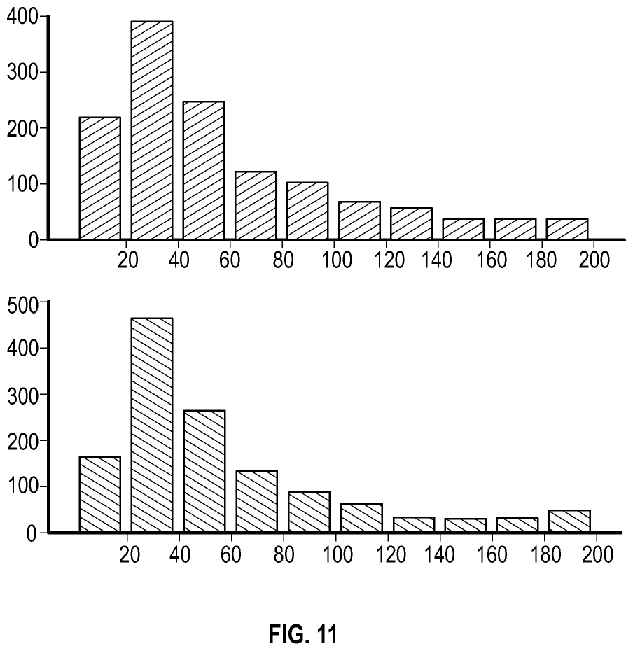

Across the two samples, 1314 metabolites were detected. A volcano plot illustrating the differences in metabolites extracted between the two workflows is show in FIG. 9. As illustrated in FIG. 9, the two methods resulted in very similar metabolic profiles. Out of 1314 metabolites detected, 92 were lower in BA with 9 significantly lower and 162 were higher with 28 significantly higher as compared to those detected in the sample processed with MTBE. To further investigate reproducibility between samples, a scores plot of the BA and MTBE replicates is shown in FIG. 10. FIG. 10 illustrates that although MTBE and BA samples could be separated along PC2, the largest differentiation was between batch (along PC1), further suggesting little variation in the identities and relative quantities of metabolites detected in each extraction. Finally, a distribution of coefficients of variation (CVs) for detected metabolites in the MTBE (top) and BA (bottom) samples are shown in FIG. 11. The distributions are similar in average value and shape, further suggesting both solvents result in a metabolomic analysis with comparable reproducibility.

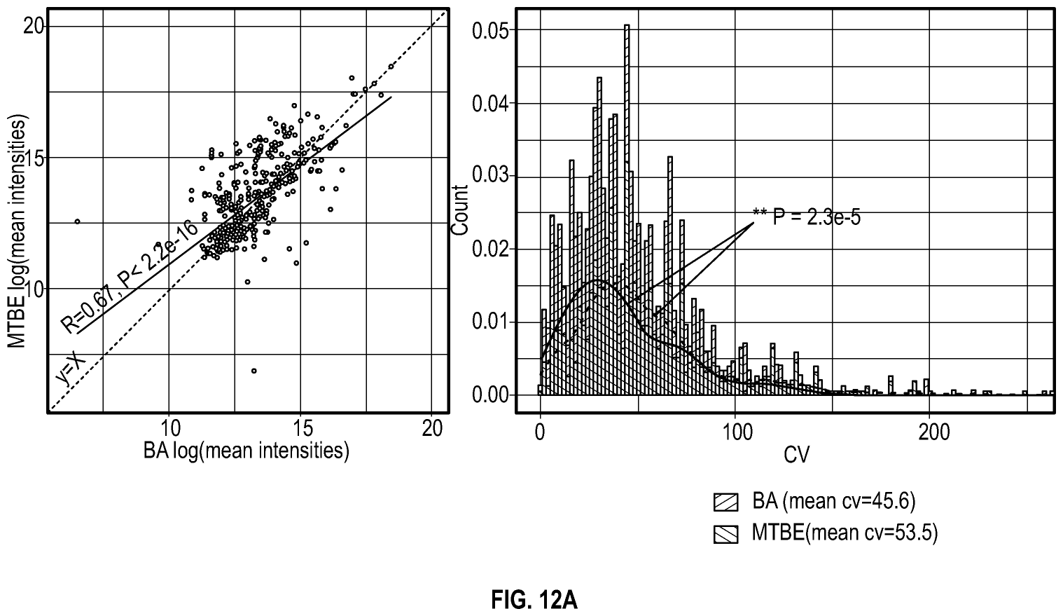

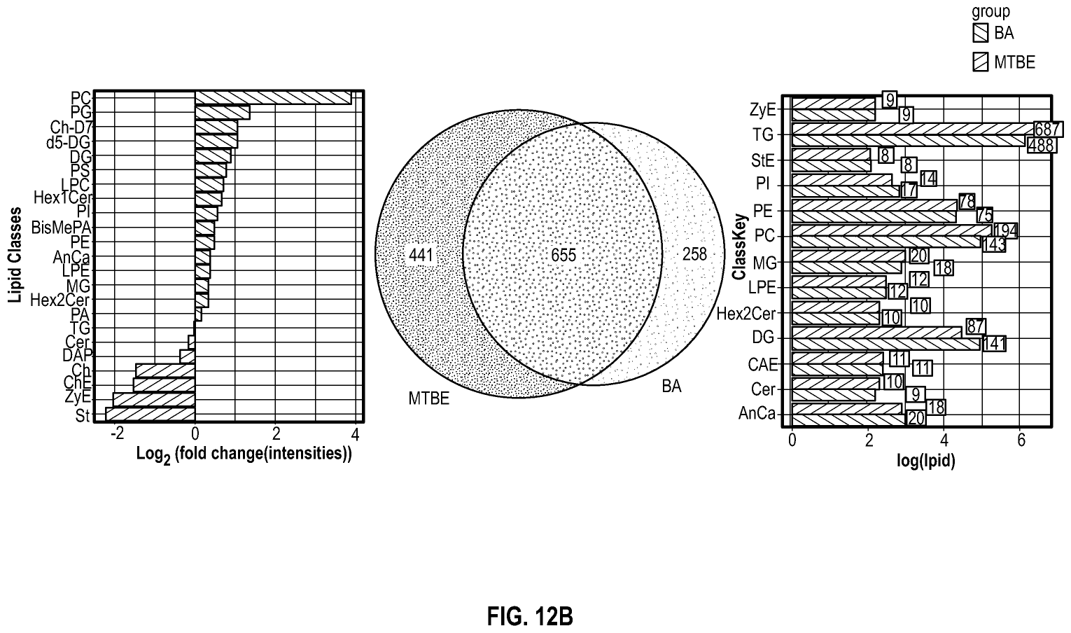

A comparison of the lipids detected in the samples processed with BA and MTBE is shown in FIGS. 12A and 12B. FIG. 12A shows a plot log mean intensities of detected lipids for BA vs. MTBE (left). The line of best-fit through the data lies below the line y=x, suggesting that intensities for lipids which were detected in both the BA and MBTE workflows showed an increased signal in the BA extraction. FIG. 12A also shows distributions of calculated coefficients of variations (CVs) for lipids identified in each for the BA and MTBE extractions (right). A statistically significant difference between the distributions was detected (p=2.5×10⁻⁵), with the distribution for the BA-extracted lipids (solid line) narrow and lower n average, suggesting extraction with BA leads to reduced variability between replicates compared to extraction with MTBE (dashed line). As illustrated in FIG. 12B (center), BA extraction resulted in the identification of 913 lipids while MTBE extraction resulted in the identification of 1,096 lipids with 655 lipids in common between the two solvents. Counts by lipid class for each solvent (MTBE, top; BA, bottom) are shown in the right panel of FIG. 12B. Finally, the log fold change of the average intensity of the detected lipids per class (ratio of BA:MTBE) are shown in the left panel of FIG. 12C. For more than half the classes detected, the BA extraction resulted in a more intense signal than the MTBE extraction.

### Example 10: Lipidomics Analysis

The different sample preparation methods for lipid extraction provided herein were compared for types and amounts of lipids extracted. Human plasma samples underwent lipid extraction by one of the following methods: 1) BUME:butyl (BUME biphasic extraction with butyl acetate), 2) BUME:heptane (BUME biphasic extraction with heptane), 3) BUME single phase extraction, 4) ethanol extraction, and 5) MTBE extraction, followed by the Lipodomics MS method as described above. BUME extractions were as described in Example 4 and MTBE extractions were as described in Example 3 with the addition of the specified organic solvent. To compare the types and amounts of lipids that were extracted by the different extraction methods as well as reproducibility of each solvent system, the replicates were further analyzed by principal component analysis (PCA).

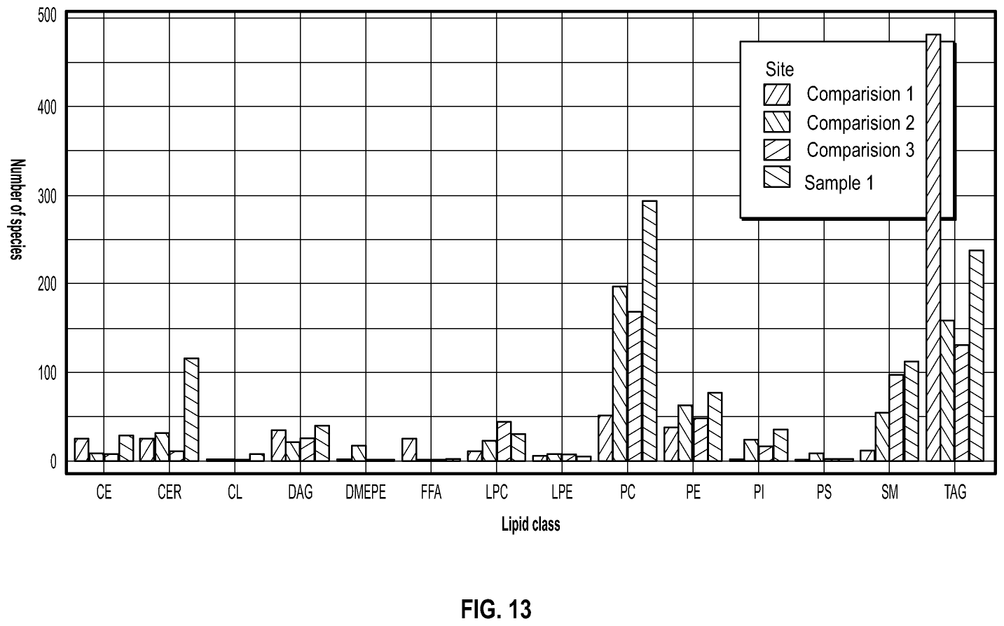

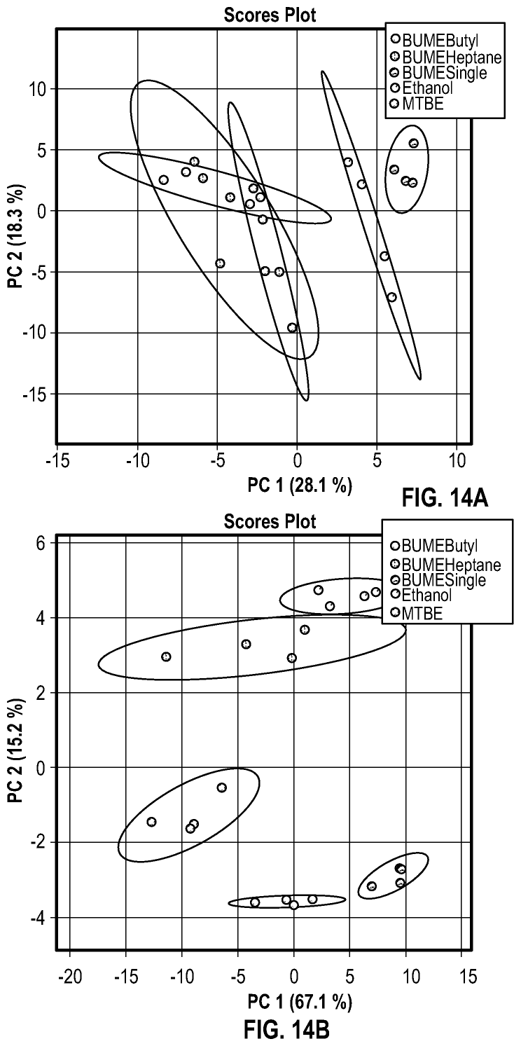

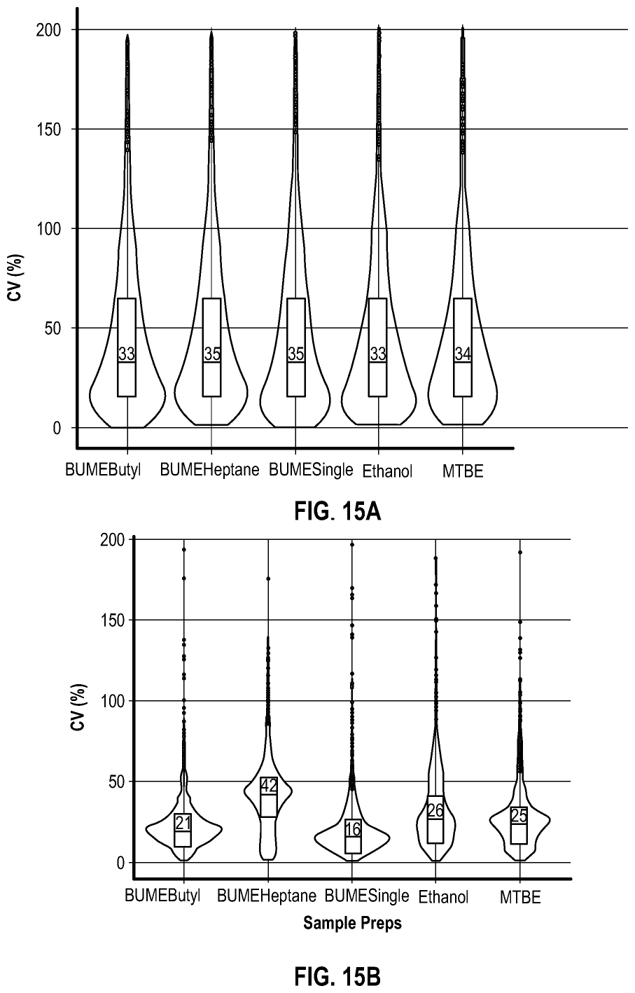

In one set of experiments (Comparison 3), 1383 lipid species (including adducts and isomers) were identified across 19 classes 573 lipids were identified in the four frozen plasma samples (FIG. 13). FIG. 14A shows a two-dimensional principal component analysis (PCA) plot of the extraction replicates with PC1 accounting for about 28.1% of the variation and PC2 of about 18.3% of the variation. Generally, BUME:butyl, BUME:heptane, and MTBE extraction methods showed similar clustering while BUME and ethanol single phase methods were mutually separated from other solvent systems. Biphasic extraction methods with butyl acetate, heptane, and methyl tert-butyl ether clustered closely together in the scores plot, further suggesting they were extracting similar lipids as one another. FIG. 15A shows coefficient of variation (CV) distribution violin plots across all lipids for the different extraction methods. Generally, all tested extraction methods had similar CVs, with a median CV ranging around 33%-35% and comparable CV distribution shape.

In a second set of experiments, 1123 lipid species (including adducts and isomers included) were identified across 17 classes (Sample 1; FIG. 13). FIG. 14B shows a 2D PCA plot of the extraction replicates with PC1 accounting for about 67.1% of the variation and PC2 of about 15.2% variation. Generally, replicates of each individual extraction solvent system clustered in the scores plot, indicating good reproducibility between replicates. Further, individual solvent systems formed mutually distinguishable clusters in the scores plot. FIG. 15B shows CV distribution violin plots across all lipids for the different extraction methods. Compared to the CV distributions in the first set of experiments (FIG. 15A), the CV distributions have lower medians and narrower shapes, suggesting reduced variability compared the first set of experiments, with the exception of the BUME:heptane system (median CV of 21% for BUME:butyl, 42% for BUME:heptane, 16% for BUME single phase extraction, 26% for ethanol extraction, and 25% for MTBE extraction).

### Example 11: Metabolomics Analysis

For a subset of the experiments (BUMEButyl, BUMEHeptane, Methanol, MTBE) discussed in Example 10, the aqueous layer was subjected to further targeted mass spectrometry (MS/MS) analysis to identify polar metabolites.

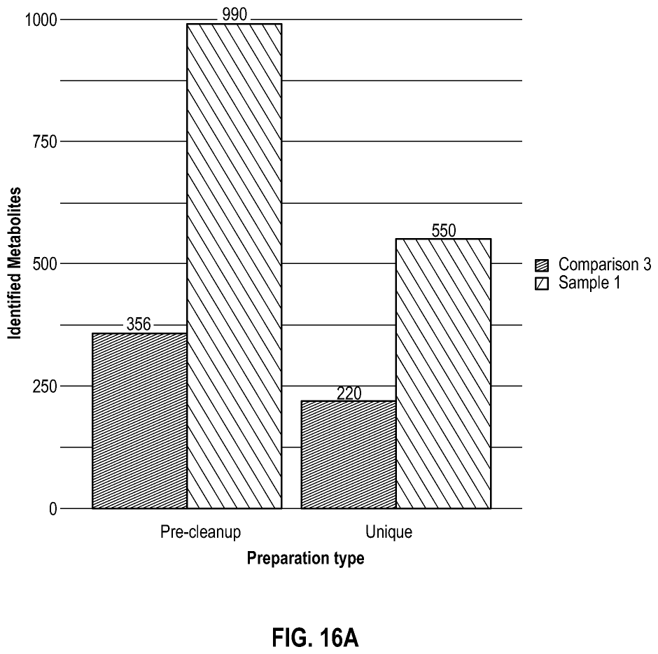

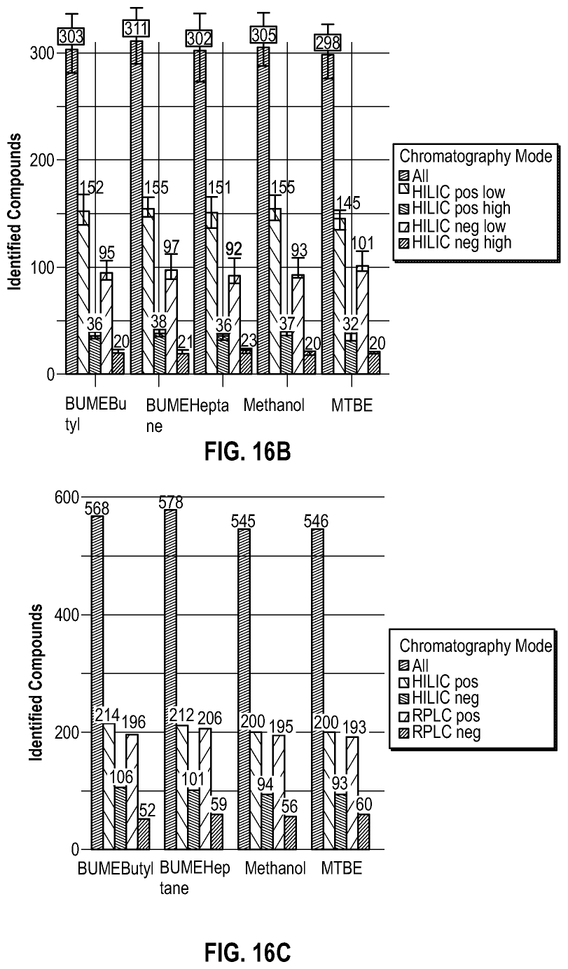

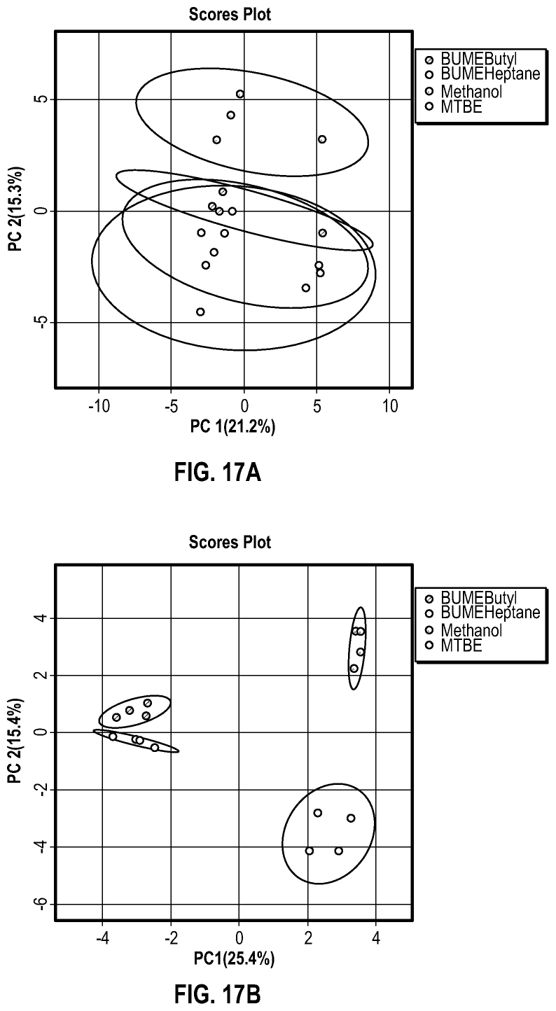

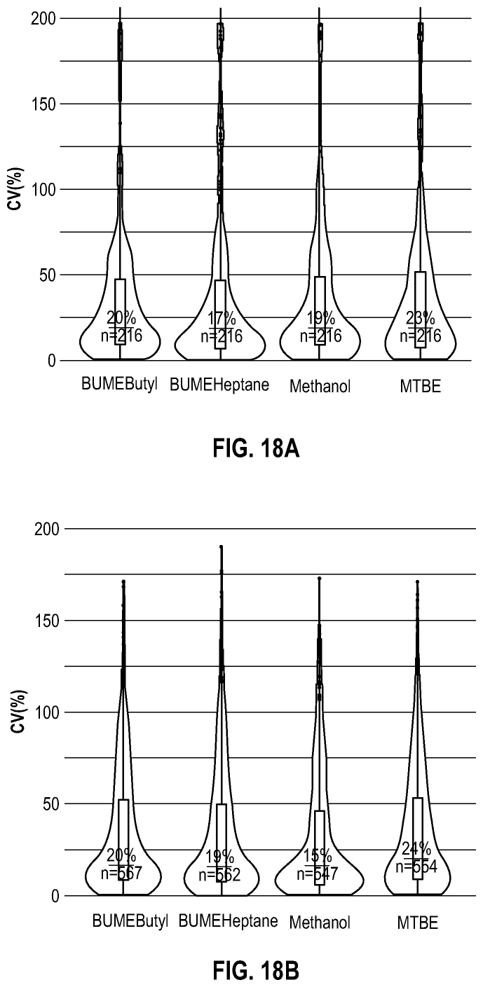

In the samples from the first set of experiments (comparison 3), 356 individual species were identified corresponding to 220 unique metabolites (FIG. 16A). The number of individual compounds identified by solvent system and chromatography mode are further detailed in FIGS. 16B and 16C. FIG. 17A shows a two-dimensional principal component analysis (PCA) plot of the replicates from the first set of experiments with PC1 accounting for about 21.2% of the variation and PC2 of about 15.3% of the variation. The extraction methods generally clustered together, with some separation among the different solvent systems along PC2. FIG. 18A shows coefficient of variation (CV) distribution violin plots across all metabolites for the different extraction methods. Generally, all tested extraction methods had similar CV, with a median CV ranging around 17%-23% and comparable CV distribution shape.

In the samples from the first set of experiments (comparison 3), 356 individual species were identified corresponding to 220 unique metabolites (FIG. 16A). The number of individual compounds identified by solvent system and chromatography mode are further detailed in FIGS. 16B and 16C. FIG. 17B shows a two-dimensional principal component analysis (PCA) plot of the replicates from the first set of experiments with PC1 accounting for about 25.4% of the variation and PC2 of about 15.4% of the variation. The extraction methods generally clustered into mutually distinct clusters. The BUME:butyl and BUME:heptane clusters are close to one another in scores space, suggesting the two solvent systems behave similarly with respect to metabolite extraction. FIG. 18B shows coefficient of variation (CV) distribution violin plots across all metabolites for the different extraction methods. Generally, all tested extraction methods had similar CV, with a median CV ranging around 15%-24% and comparable CV distribution shape.

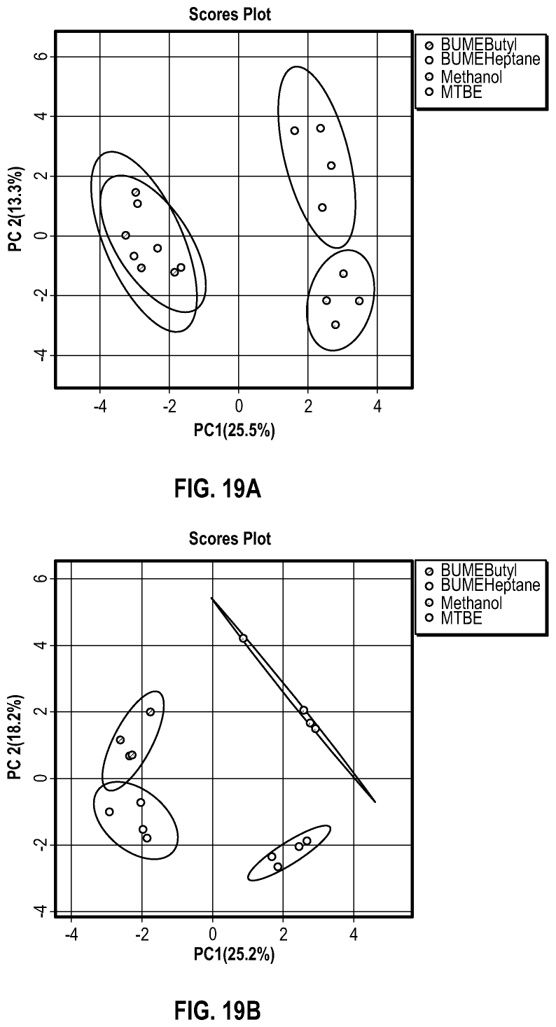

PCA was additionally performed on subsets of the samples from the second set of experiments corresponding to the differing chromatographic modes employed (hydrophobic interaction chromatography (HILIC) and reverse phase liquid chromatography (RPLC)). In each case, the first principal component was found to explain about 25% of the variance across replicates and the second principal component was found to explain about 13%-18% of the variance (FIGS. 19A and 19B).

For the HILIC replicates (FIG. 19A), the BUMEbutyl and BUMEheptane replicates were found to coincide in the scores plot, suggesting these two systems extracted substantially the same sets of metabolites. The remaining two systems each individually clustered separately. For the RPLC replaces (FIG. 19B), the replicates separated into substantially mutually distinct groups, though the two BUME systems were again closest in scores space.

### List of Embodiments

The following list of numbered embodiments of the invention are to be considered as disclosing various features of the invention, which features can be considered to be specific to the particular embodiment under which they are discussed, or which are combinable with the various other features listed in other embodiments. Thus, simply because a feature is discussed under one particular embodiment does not necessarily limit the use of that feature to that embodiment.

Embodiment 1. A method of assaying a biological sample, comprising: (a) providing a solution comprising a plurality of biomolecules, wherein the plurality of biomolecules comprises at least one protein and at least one lipid; (b) contacting the solution with a first surface, thereby capturing a first subset of biomolecules in the plurality of biomolecules on the first surface, wherein the subset of biomolecules comprises the at least one protein; (c) collecting a portion of the solution comprising at least a subset of non-captured biomolecules in the plurality of biomolecules; (d) contacting the portion of the solution with a second surface, thereby capturing a second subset of biomolecules in the subset of non-captured biomolecules, wherein the second subset of biomolecules comprises the at least one lipid; (e) releasing the at least one lipid from the second surface; and (f) performing mass spectrometry on the at least one lipid, thereby identifying the at least one lipid. Embodiment 2. The method of embodiment 1, wherein the contacting in (b) comprises adding a crashing solvent to the plurality of biomolecules.

Embodiment 3. The method of embodiment 2, wherein the crashing solvent crashes out the at least one protein onto the one or more surfaces. Embodiment 4. The method of embodiment 1, wherein the at least one lipid comprises a chylomicron.

Embodiment 5. A method of assaying a biological sample, comprising: (a) providing the biological sample comprising a plurality of biomolecules, wherein the plurality of biomolecules comprises at least one protein and at least one lipid or metabolite; (b) contacting the biological sample with a surface to bind the at least one protein, thereby yielding an altered composition of the biological sample, wherein the altered composition of the biological sample comprises a reduced amount of the at least one protein; (c) adding a first solvent to the altered composition of the biological sample to generate a mixture comprising a first organic layer and an aqueous layer, wherein the at least one lipid or metabolite partitions into the first organic layer; and (d) performing mass spectrometry on at least a portion of the first organic layer, thereby identifying the at least one lipid or metabolite. Embodiment 6. The method of embodiment 5, wherein (b) further comprises separating the surface and the altered composition of the biological sample.

Embodiment 7. A method of assaying a biological sample, comprising: (a) providing the biological sample comprising a plurality of biomolecules, wherein the plurality of biomolecules comprises at least one protein and at least one lipid or metabolite; (b) adding a first solvent to the biological sample to generate a mixture comprising a first organic layer and an aqueous layer, wherein the at least one lipid or metabolite partitions into the first organic layer; (c) contacting at least a portion of the first organic layer or the aqueous layer with a surface, wherein the at least one protein binds to the surface; and (d) performing mass spectrometry on at least a portion of the first organic layer, thereby identifying the at least one lipid or metabolite. Embodiment 8. The method of embodiment 7, further comprising, after the contacting, separating the first organic layer or the aqueous layer from the surface.

Embodiment 9. A method of assaying a biological sample, comprising:
(a) providing the biological sample comprising a plurality of biomolecules, wherein the plurality of biomolecules comprises at least one lipid or metabolite; (b) contacting at least a portion of the sample with a surface to adsorb the at least one lipid or metabolite on the surface; (c) separating the at least one lipid or metabolite from the surface to generate an isolated lipid or metabolite; and (d) performing mass spectrometry on the isolated lipid or metabolite, thereby identifying the at least one lipid or metabolite. Embodiment 10. The method of embodiment 9, wherein the separating comprises contacting the at least one lipid or metabolite with a first solvent. Embodiment 11. The method of embodiment 10, wherein the biological sample further comprises at least one protein. Embodiment 12. The method of embodiment 11, further comprising performing mass spectrometry on the at least one protein. Embodiment 13. The method of embodiment 11 or embodiment 12, further comprising contacting at least another portion of the sample with a second surface, wherein the second surface is configured to bind the at least one protein. Embodiment 14. The method of embodiment 13, wherein the contacting the at least another portion of the sample with a second surface is performed prior to the contacting in (b). Embodiment 15. The method of embodiment 13, wherein the contacting the at least another portion of the sample with the second surface is performed subsequent to the contacting in (b). Embodiment 16. The method of any one of embodiments 5-15, further comprising adding water to the biological sample. Embodiment 17. The method of any one of embodiments 5-16, wherein a density of the first solvent is lower than a density of water. Embodiment 18. The method of any one of embodiments 5-16, wherein a density of the first solvent is higher than a density of water. Embodiment 19. The method of any one of embodiments 5-16, wherein a dielectric constant of the first solvent is lower than a dielectric constant of water. Embodiment 20. The method of any one of embodiments 5-16, wherein the dielectric constant of the first solvent is higher than the dielectric constant of water. Embodiment 21. The method of any one of embodiments 5-16, wherein a dielectric constant of the first solvent is less than about 1, about 2, about 3, about 4, about 5, about 6, about 7, about 8, about 9, about 10, about 20, about 30, about 40, about 50, about 60, about 70, or about 80 at about 20° C. Embodiment 22. The method of any one of embodiments 5-21, wherein a viscosity of the first solvent is greater than about 0.3 mPa·s, 0.35 mPa·s, 0.4 mPa·s, 0.45 mPa·s, 0.5 mPa·s, 0.55 mPa·s, 0.6 mPa·s, 0.65 mPa·s, 0.7 mPa·s, 0.75 mPa·s, or 0.8 mPa·s. Embodiment 23. The method of any one of embodiments 5-22, wherein a vapor pressure of the first solvent is less than about 400, about 300, about 250, about 200, about 100, about 90, about 80, about 70, about 60, or about 50 torr at about 20° C. Embodiment 24. The method of any one of embodiments 5-23, wherein the first solvent comprises an alcohol, an aliphatic hydrocarbon, an aromatic hydrocarbon, an aliphatic heterocyclic compound, an aromatic heterocyclic compound, an amide, an ester, an ether, a ketone, a halocarbon, or a nitrile, or any combination thereof. Embodiment 25. The method of embodiment 24, wherein the first solvent comprises an ester or ether. Embodiment 26. The method of embodiment 25, wherein the first solvent comprises ethyl acetate, propyl acetate, butyl acetate, or amyl acetate, or any isomer or combination thereof. Embodiment 27. The method of embodiment 26, wherein the first solvent comprises butyl acetate. Embodiment 28. The method of any one of embodiments 25-27, wherein the first solvent comprises diethyl ether, methyl tert-butyl ether (MTBE), dioxane, tetrahydrofuran (THF), or anisole, or any isomer or combination thereof. Embodiment 29. The method of embodiment 28, wherein the first solvent comprises MTBE. Embodiment 30. The method of any one of embodiments 24-29, wherein the first solvent does not comprise a halocarbon. Embodiment 31. The method of any one of embodiments 24-30, wherein the first solvent comprises an alcohol. Embodiment 32. The method of embodiment 31, wherein the first solvent comprises methanol, ethanol, propanol, butanol, pentanol, or hexanol, or any isomer or combination thereof. Embodiment 33. The method of embodiment 32, wherein the first solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. Embodiment 34. The method of embodiment 32, wherein the first solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. Embodiment 35. The method of any one of embodiments 5-34, further comprising adding a second solvent to the altered composition of the biological sample, the first organic layer, or the aqueous layer. Embodiment 36. The method of embodiment 35, wherein the second solvent substantially partitions into the first organic layer. Embodiment 37. The method of embodiment 35, wherein the second solvent substantially partitions into a second organic layer. Embodiment 38. The method of any one of embodiments 35-37, wherein the first solvent comprises an alcohol and the second solvent comprises an ester. Embodiment 39. The method of embodiment 38, wherein the ester comprises butyl acetate. Embodiment 40. The method of embodiment 38 or embodiment 39, wherein the alcohol comprises butanol, methanol, or a combination thereof. Embodiment 41. The method of any one of embodiments 35-40, wherein the first solvent comprises an alcohol and the second solvent comprises an ester and an ether. Embodiment 42. The method of embodiment 41, wherein the ether comprises methyl tert-butyl ether (MTBE). Embodiment 43. The method of embodiment 41, or embodiment 42, wherein the ester comprises butyl acetate. Embodiment 44. The method of any one of embodiments 5-43, further comprising adding a pH adjusting agent to the altered composition of the biological sample. Embodiment 45. The method of embodiment 44, wherein the pH adjusting agent comprises a base. Embodiment 46. The method of embodiment 44 or embodiment 45, wherein the pH adjusting agent comprises an acid. Embodiment 47. The method of any one of embodiments 5-46, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. Embodiment 48. The method of embodiment 47, wherein the biological sample comprises blood, serum, or plasma, or any portion of fraction thereof. Embodiment 49. The method of embodiment 48, wherein the biological sample comprises plasma. Embodiment 50. The method of embodiment 49, wherein the plasma is diluted. Embodiment 51. The method of any one of embodiments 5-50, further comprising purifying the at least one lipid or metabolite from the first organic layer. Embodiment 52. The method of any one of embodiments 5-51, wherein the at least one lipid comprises a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a sphingomyelin (SM), a cardiolipin (CL), or a free fatty acid (FFA), or any derivative thereof. Embodiment 53. The method of embodiment 52, wherein the at least one lipid comprises a ChE, a CER, a CL, a DAG, a LPC, a LPE, a PG, a PE, a PI, a SM, or a TAG. Embodiment 54. The method of any one of embodiments 5-53, further comprising performing mass spectrometry on the at least one protein. Embodiment 55. The method of embodiment 54, further comprising, prior to performing mass spectrometry on the at least one protein, separating the at least one protein from the surface. Embodiment 56. The method of embodiment 54 or embodiment 55, further comprising lysing the at least one protein. Embodiment 57. The method of embodiment 56, further comprising, digesting the at least one protein to generated digested peptides. Embodiment 58. The method of embodiment 57, further comprising purifying the digested peptides. Embodiment 59. The method of any one of embodiments 5-58, further comprising performing mass spectrometry on at least a portion of the aqueous layer to identify at least another metabolite present in the biological sample. Embodiment 60. The method of any one of embodiments 5-59, wherein the at least one lipid is comprised in a plurality of lipids in the biological sample. Embodiment 61. The method of embodiment 60, wherein the plurality of lipids comprises a dynamic range of at least 1, 2, 3, 4, 5, 6, 7, 8, 9, 10, 11, 12, 13, or 14 orders of magnitude in the biological sample. Embodiment 62. The method of embodiment 60, wherein the plurality of lipids comprises a dynamic range of no more than about 14, 13, 12, 11, 10, 9, 8, 7, 6, 5, 4, 3, 2, or 1 orders of magnitude in the biological sample. Embodiment 63. The method of any one of embodiments 60-62, wherein at least a portion of the plurality of lipid comprises a dynamic range in the first organic layer which is less than a dynamic range of the plurality of lipids in the biological sample. Embodiment 64. The method of any one of embodiments 5-63, wherein the at least one metabolite comprises a vitamin, a cofactor, a nucleotide, a polynucleotide, an amino acid or analogue thereof, a peptidomimetic, an organic acid, an alcohol, a diol, a polyol, a monosaccharide, a disaccharide, an oligosaccharide, a polysaccharide, a hydrocarbon, a benzenoid, an alkaloid, an acetylide, a polyketide, a terpene or terpenoid, a phenolic, or any combination or derivative thereof. Embodiment 65. The method of any one of embodiments 5-64, wherein the first surface is a surface of a particle. Embodiment 66. The method of embodiment 65, wherein the particle is a nanoparticle. Embodiment 67. The method of embodiment 65, wherein the particle is a microparticle. Embodiment 68. The method of any one of embodiments 65-67, wherein the particle is a micelle, liposome, iron oxide particle, silver particle, gold particle, palladium particle, quantum dot, platinum particle, titanium particle, silica particle, metal or inorganic oxide particle, synthetic polymer particle, copolymer particle, terpolymer particle, polymeric particle with metal cores, polymeric particle with metal oxide cores, polystyrene sulfonate particle, polyethylene oxide particle, polyoxyethylene glycol particle, polyethylene imine particle, polylactic acid particle, polycaprolactone particle, polyglycolic acid particle, poly(lactide-co-glycolide polymer particle, cellulose ether polymer particle, polyvinylpyrrolidone particle, polyvinyl acetate particle, polyvinylpyrrolidone-vinyl acetate copolymer particle, polyvinyl alcohol particle, acrylate particle, polyacrylic acid particle, crotonic acid copolymer particle, polyethlene phosphonate particle, polyalkylene particle, carboxy vinyl polymer particle, sodium alginate particle, carrageenan particle, xanthan gum particle, gum acacia particle, Arabic gum particle, guar gum particle, pullulan particle, agar particle, chitin particle, chitosan particle, pectin particle, karaya tum particle, locust bean gum particle, maltodextrin particle, amylose particle, corn starch particle, potato starch particle, rice starch particle, tapioca starch particle, pea starch particle, sweet potato starch particle, barley starch particle, wheat starch particle, hydroxypropylated high amylose starch particle, dextrin particle, levan particle, elsinan particle, gluten particle, collagen particle, whey protein isolate particle, casein particle, milk protein particle, soy protein particle, keratin particle, polyethylene particle, polycarbonate particle, polyanhydride particle, polyhydroxyacid particle, polypropylfumerate particle, polycaprolactone particle, polyamine particle, polyacetal particle, polyether particle, polyester particle, poly(orthoester) particle, polycyanoacrylate particle, polyurethane particle, polyphosphazene particle, polyacrylate particle, polymethacrylate particle, polycyanoacrylate particle, polyurea particle, polyamine particle, polystyrene particle, poly(lysine) particle, chitosan particle, dextran particle, poly(acrylamide) particle, derivatized poly(acrylamide) particle, gelatin particle, starch particle, chitosan particle, dextran particle, gelatin particle, starch particle, poly-β-amino-ester particle, poly(amido amine) particle, poly lactic-co-glycolic acid particle, polyanhydride particle, bioreducible polymer particle, 2-(3-aminopropylamino) ethanol particle, protein functionalized particle, ubiquitin functionalized particle, polysaccharide coated particle, or dextran functionalized particle. Embodiment 69. The method of any one of embodiments 65-68, wherein the particle is a magnetic particle. Embodiment 70. The method of embodiment 69, wherein the magnetic particle is a superparamagnetic iron oxide particle. Embodiment 71. The method of any one of embodiments 65-68, wherein the particle comprises an iron oxide material. Embodiment 72. The method of embodiment 71, wherein the particle comprises an iron oxide core. Embodiment 73. The method of embodiment 71, wherein the particle comprises iron oxide crystals embedded in a polystyrene core. Embodiment 74. The method of any one of embodiments 65-73, wherein the particle comprises a polymer coating. Embodiment 75. The method of embodiment 74, wherein the particle comprises a positively charged polymer, a negatively charged polymer, a zwitterionic polymer, or any combination thereof. Embodiment 76. The method of any one of embodiments 65-75, wherein the particle a silica shell coating. Embodiment 77. The method of any one of embodiments 65-75, wherein the particle comprises a poly(N-(3-(dimethylamino)propyl) methacrylamide) (PDMAPMA) coating. Embodiment 78. The method of any one of embodiments 65-75, wherein the particle comprises a poly(oligo(ethylene glycol) methyl ether methacrylate) (POEGMA) coating. Embodiment 79. The method of any one of embodiments 65-78, wherein the particle comprises a positive surface charge. Embodiment 80. The method of any one of embodiments 65-78, wherein the particle comprises a negative surface charge. Embodiment 81. The method of any one of embodiments 65-78, wherein the particle comprises a neutral surface charge. Embodiment 82. The method of any one of embodiments 5-81, wherein the contacting comprises contacting the sample, the first organic layer, or the aqueous layer with a plurality of surface regions. Embodiment 83. The method of embodiment 82, wherein the plurality of surface regions is disposed on the first surface. Embodiment 84. The method of embodiment 82, wherein the plurality of surface regions are disposed on a plurality of discrete surfaces comprising the first surface. Embodiment 85. The method of any one of embodiments 82-84, wherein a first surface region of the plurality of surface regions comprises a first physiochemical property and a second surface region of the plurality of surface regions comprises a second physicochemical property different from the first physicochemical property. Embodiment 86. The method of embodiment 85, wherein the first physicochemical property comprises charge, zeta potential, hydrophobicity, surface functional group, or any combination thereof. Embodiment 87. The method of embodiment 86, wherein the plurality of discrete surfaces are surfaces of a plurality of particles. Embodiment 88. The method of embodiment 87, wherein the first physicochemical property comprises size, charge, core material, shell material, porosity, zeta potential, hydrophobicity, surface functional group, or any combination thereof. Embodiment 89. The method of any one of embodiments 82-88, wherein the plurality of surface regions is comprised on an array. Embodiment 90. The method of any one of embodiments 5-89, wherein the biological sample comprises at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids or metabolites. Embodiment 91. The method of any one of embodiments 5-89, wherein the biological sample comprises no more than about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites. Embodiment 92. The method of any one of embodiments 5-91, wherein the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids or metabolites. Embodiment 93. The method of embodiment 92, wherein the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids. Embodiment 94. The method of embodiment 92 or embodiment 93, wherein the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct metabolites. Embodiment 95. The method of any one of embodiments 5-91, wherein the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids or metabolites. Embodiment 96. The method of embodiment 95, wherein the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids. Embodiment 97. The method of embodiment 95 or embodiment 96, wherein the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct metabolites. Embodiment 98. The method of any one of embodiments 5-97, wherein the performing mass spectrometry comprises identifying at least about 10, 20, 30, 40, 50, 60, 70, 80, 90, 100, or more cholesteryl esters (ChEs) in the biological sample. Embodiment 99. The method of any one of embodiments 5-98, wherein the performing mass spectrometry comprises identifying at least about 10, 20, 30, 40, 50, 60, 70, 80, 90, 100, 110, 120, 130, 140, 150, 160, 170, 180, 190, 200, or more ceramides (CERs) in the biological sample. Embodiment 100. The method of any one of embodiments 5-99, wherein the performing mass spectrometry comprises identifying at least about 10, 20, 30, 40, 50, 60, 70, 80, 90, 100, or more cardiolipins (CLs) in the biological sample. Embodiment 101. The method of any one of embodiments 5-100, wherein the performing mass spectrometry comprises identifying at least about 10, 20, 30, 40, 50, 60, 70, 80, 90, 100, or more diacyl glycerols (DAGs) in the biological sample. Embodiment 102. The method of any one of embodiments 5-101, wherein the performing mass spectrometry comprises identifying at least about 10, 20, 30, 40, 50, 60, 70, 80, 90, 100, or more lysophosphatidylcholines (LPCs) in the biological sample. Embodiment 103. The method of any one of embodiments 5-102, wherein the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more phosphatidylcholines (PCs) in the biological sample. Embodiment 104. The method of any one of embodiments 5-103, wherein the performing mass spectrometry comprises identifying at least about 10, 20, 30, 40, 50, 60, 70, 80, 90, 100, 110, 120, 130, 140, 150, 160, 170, 180, 190, 200, or more phosphatidylethanolamines (PEs) in the biological sample. Embodiment 105. The method of any one of embodiments 5-104, wherein the performing mass spectrometry comprises identifying at least about 10, 20, 30, 40, 50, 60, 70, 80, 90, 100, or more phosphatidylinositols (PIs) in the biological sample. Embodiment 106. The method of any one of embodiments 5-105, wherein the performing mass spectrometry comprises identifying at least about 10, 20, 30, 40, 50, 60, 70, 80, 90, 100, 110, 120, 130, 140, 150, 160, 170, 180, 190, 200, or more sphingomyelins (SMs) in the biological sample. Embodiment 107. The method of any one of embodiments 5-106, wherein the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more triacylglycerols (TAGs) in the biological sample.

Embodiment 108. A method of assaying a biological sample, comprising: (a) providing the biological sample comprising a plurality of biomolecules, wherein the plurality of biomolecules comprises at least one lipid; (b) adding a first solvent to the biological sample to generate a mixture comprising a first organic layer and an aqueous layer, wherein the first organic layer comprises the at least one lipid, and wherein the first solvent comprises butyl acetate; and (c) performing mass spectrometry on the first organic layer, thereby identifying the at least one lipid. Embodiment 109. The method of embodiment 108, wherein the biological sample further comprises at least one protein. Embodiment 110. The method of embodiment 109, further comprising, contacting the biological sample, the first organic layer, or the aqueous layer with a surface, wherein the at least one protein binds to the surface. Embodiment 111. The method of embodiment 109 or embodiment 110, further comprising performing mass spectrometry on the at least one protein. Embodiment 112. The method of embodiment 110 or embodiment 111, wherein the first surface is a surface of particle. Embodiment 113. The method of embodiment 112, wherein the particle is a nanoparticle. Embodiment 114. The method of embodiment 113, wherein the particle is a microparticle. Embodiment 115. The method of any one of embodiments 110-114, wherein the contacting comprises contacting the sample, the first organic layer, or the aqueous layer with a plurality of surface regions. Embodiment 116. The method of embodiment 115, wherein the plurality of surface regions is disposed on the surface. Embodiment 117. The method of embodiment 115, wherein the plurality of surface regions are disposed on a plurality of discrete surfaces comprising the surface. Embodiment 118. The method of any one of embodiments 115-117, wherein first surface region of the plurality of surface regions comprises a first physiochemical property and a second surface region of the plurality of surface regions comprises a second physicochemical property different from the first physicochemical property. Embodiment 119. The method of embodiment 118, wherein the first physicochemical property comprises charge, zeta potential, hydrophobicity, surface functional group, or any combination thereof. Embodiment 120. The method of embodiment 119, wherein the plurality of discrete surfaces are surfaces of a plurality of particles. Embodiment 121. The method of embodiment 120, wherein the wherein the first physicochemical property comprises size, charge, core material, shell material, porosity, zeta potential, hydrophobicity, surface functional group, or any combination thereof. Embodiment 122. The method of any one of embodiments 110-121, wherein the plurality of surface regions is comprised on an array. Embodiment 123. The method of any one of embodiments 108-122, further comprising adding water to the biological sample. Embodiment 124. The method of any one of embodiments 108-123, wherein the first solvent further comprises an alcohol, an aliphatic hydrocarbon, an aromatic hydrocarbon, an aliphatic heterocyclic compound, an aromatic heterocyclic compound, an amide, an ester, an ether, a ketone, a halocarbon, or a nitrile, or any combination thereof. Embodiment 125. The method of embodiment 124, wherein the first solvent comprises an ester. Embodiment 126. The method of any one of embodiments 125, wherein the first solvent comprises diethyl ether, methyl tert-butyl ether (MTBE), dioxane, tetrahydrofuran (THF), or anisole, or any isomer or combination thereof. Embodiment 127. The method of embodiment 126, wherein the first solvent comprises MTBE. Embodiment 128. The method of any one of embodiments 108-127, wherein the first solvent does not comprise a halocarbon. Embodiment 129. The method of any one of embodiments 108-128, wherein the first solvent comprises an alcohol. Embodiment 130. The method of embodiment 129, wherein the first solvent comprises methanol, ethanol, propanol, butanol, pentanol, or hexanol, or any isomer or combination thereof. Embodiment 131. The method of embodiment 130, wherein the first solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. Embodiment 132. The method of embodiment 130, wherein the first solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. Embodiment 133. The method of any one of embodiments 108-132, further comprising adding a second solvent to the biological sample, the first organic layer or the aqueous layer. Embodiment 134. The method of embodiment 133, wherein the second solvent substantially partitions into the first organic layer. Embodiment 135. The method of embodiment 133, wherein the second solvent substantially partitions into a second organic layer. Embodiment 136. The method of any one of embodiments 133-135, wherein the second solvent comprises an alcohol. Embodiment 137. The method of embodiment 136, wherein the second solvent comprises methanol, ethanol, propanol, butanol, pentanol, or hexanol, or any isomer or combination thereof. Embodiment 138. The method of embodiment 137, wherein the second solvent comprises a mixture of butanol and methanol comprising a volume/volume (v/v) ratio of at least about 1:50, 1:20, 1:10, 1:5, 1:4, 1:3, 1:2, 1:1, 2:1, 3:1, 4:1, 5:1, 10:1, 20:1, 50:1, or more. Embodiment 139. The method of embodiment 137, wherein the second solvent comprises a mixture of butanol and methanol comprising a volume/volume (v/v) ratio of no more than about 50:1, 20:1, 10:1, 5:1, 4:1, 3:1, 2:1, 1:1, 1:2, 1:3, 1:4, 1:5, 1:10, 1:20, 1:50, or less. Embodiment 140. The method of any one of embodiments 133-139, wherein the second solvent comprises an ester. Embodiment 141. The method of embodiment 140, wherein the second solvent comprises methyl tert-butyl ether (MTBE). Embodiment 142. The method of any one of embodiments 108-141, further comprising adding a pH adjusting agent to the biological sample, the first organic layer, the second organic layer, or the aqueous layer. Embodiment 143. The method of embodiment 142, wherein the pH adjusting agent comprises a base. Embodiment 144. The method of embodiment 142 or embodiment 143, wherein the pH adjusting agent comprises an acid. Embodiment 145. The method of any one of embodiments 108-144, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. Embodiment 146. The method of embodiment 145, wherein the biological sample comprises blood, serum, or plasma, or any portion of fraction thereof. Embodiment 147. The method of embodiment 146, wherein the biological sample comprises plasma. Embodiment 148. The method of any one of embodiments 108-147, further comprising purifying the at least one lipid from the first organic layer. Embodiment 149. The method of any one of embodiments 108-148, wherein the at least one lipid comprises a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a cardiolipin (CL), or a sphingomyelin (SM), or any derivative thereof. Embodiment 150. The method of embodiment 149, wherein the at least one lipid comprises a ChE, a CER, a CL, a DAG, a LPC, a LPE, a PG, a PE, a PI, a SM, or a TAG. Embodiment 151. The method of any one of embodiments 108-150, wherein the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids. Embodiment 152. The method of any one of embodiments 108-150, wherein the performing mass spectrometry identifies at most about 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or fewer distinct lipids. Embodiment 153. The method of any one of embodiments 108-152, wherein the performing mass spectrometry comprises identifying at least about 10, 20, 30, 40, 50, 60, 70, 80, 90, 100, or more cholesteryl esters (ChEs) in the biological sample. Embodiment 154. The method of any one of embodiments 108-153, wherein the performing mass spectrometry comprises identifying at least about 10, 20, 30, 40, 50, 60, 70, 80, 90, 100, 110, 120, 130, 140, 150, 160, 170, 180, 190, 200, or more ceramides (CERs) in the biological sample. Embodiment 155. The method of any one of embodiments 108-154, wherein the performing mass spectrometry comprises identifying at least about 10, 20, 30, 40, 50, 60, 70, 80, 90, 100, or more cardiolipins (CLs) in the biological sample. Embodiment 156. The method of any one of embodiments 108-155, wherein the performing mass spectrometry comprises identifying at least about 10, 20, 30, 40, 50, 60, 70, 80, 90, 100, or more diacyl glycerols (DAGs) in the biological sample. Embodiment 157. The method of any one of embodiments 108-156, wherein the performing mass spectrometry comprises identifying at least about 10, 20, 30, 40, 50, 60, 70, 80, 90, 100, or more lysophosphatidylcholines (LPCs) in the biological sample. Embodiment 158. The method of any one of embodiments 108-157, wherein the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more phosphatidylcholines (PCs) in the biological sample. Embodiment 159. The method of any one of embodiments 108-158, wherein the performing mass spectrometry comprises identifying at least about 10, 20, 30, 40, 50, 60, 70, 80, 90, 100, 110, 120, 130, 140, 150, 160, 170, 180, 190, 200, or more phosphatidylethanolamines (PEs) in the biological sample. Embodiment 160. The method of any one of embodiments 108-159, wherein the performing mass spectrometry comprises identifying at least about 10, 20, 30, 40, 50, 60, 70, 80, 90, 100, or more phosphatidylinositols (PIs) in the biological sample. Embodiment 161. The method of any one of embodiments 108-160, wherein the performing mass spectrometry comprises identifying at least about 10, 20, 30, 40, 50, 60, 70, 80, 90, 100, 110, 120, 130, 140, 150, 160, 170, 180, 190, 200, or more sphingomyelins (SMs) in the biological sample. Embodiment 162. The method of any one of embodiments 108-161, wherein the performing mass spectrometry comprises identifying at least about 10, 20, 30, 40, 50, 60, 70, 80, 90, 100, 110, 120, 130, 140, 150, 160, 170, 180, 190, 200, 300, 400, 500, 600, 700, 800, 900, 1,000, or more triacylglycerols (TAGs) in the biological sample.

Embodiment 163. An apparatus for assaying a biological sample comprising at least one protein and at least one lipid or metabolite, the apparatus comprising: a substrate comprising a surface; a loading unit that is operably coupled to the substrate; and a computer-readable medium comprising machine-executable code that, upon execution by one or more processors programmed individually or collectively, implements a method comprising: (i) transferring at least a portion of the biological sample to the surface, thereby contacting the at least the portion of the biological sample with the surface to bind the at least one protein and yield an altered composition of the at least the portion of the biological sample, wherein the altered composition of the biological sample comprises a reduced amount of the at least one protein; and (ii) adding a first solvent to at least a portion of the altered composition of the biological sample to generate a mixture comprising a first organic layer and an aqueous layer, wherein the at least one lipid or metabolite partitions into the first organic layer. Embodiment 164. The apparatus of embodiment 163, wherein the method further comprises transferring at least a portion of the mixture to a container. Embodiment 165. The apparatus of embodiment 164, wherein the at least a portion of the mixture comprises at least a portion of the organic layer. Embodiment 166. The apparatus of embodiment 164 or embodiment 165, wherein the at least a portion of the mixture comprises at least a portion of the aqueous layer. Embodiment 167. The apparatus of any one of embodiments 163-166, wherein the first solvent comprise butyl acetate.

Embodiment 168. A kit for assaying a biological sample, comprising: a substrate comprising a surface, wherein the substrate comprises a surface configured to bind a biomolecule in the biological sample; and a first organic solvent. Embodiment 169. The kit of embodiment 168, wherein the first organic solvent comprises butyl acetate.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1.-41. (canceled)

42. A method of assaying a biological sample, comprising:
a. providing the biological sample comprising a plurality of biomolecules, wherein the plurality of biomolecules comprises at least one lipid;
b. adding a first solvent to the biological sample to generate a mixture comprising a first organic layer and an aqueous layer, wherein the first organic layer comprises the at least one lipid, and wherein the first solvent comprises butyl acetate; and
c. performing mass spectrometry on the first organic layer, thereby identifying the at least one lipid.

43. The method of claim 42, further comprising, contacting the biological sample, the first organic layer, or the aqueous layer with a surface, wherein the at least one protein binds to the surface.

44. The method of claim 42, further comprising performing mass spectrometry on the at least one protein.

45. The method of claim 42, wherein the first surface is a surface of particle.

46. The method of claim 45, wherein the particle is a nanoparticle.

47. The method of claim 45, wherein the particle is a microparticle.

48. The method of claim 42, wherein the first solvent does not comprise a halocarbon.

49. The method of claim 42, further comprising adding a second solvent to the biological sample, the first organic layer or the aqueous layer.

50. The method of claim 49, wherein the second solvent comprises methyl tert-butyl ether (MTBE).

51. The method of claim 42, further comprising adding a pH adjusting agent to the biological sample, the first organic layer, the second organic layer, or the aqueous layer.

52. The method of claim 42, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

53. The method of claim 52, wherein the biological sample comprises blood, serum, or plasma, or any portion of fraction thereof.

54. The method of claim 42, further comprising purifying the at least one lipid from the first organic layer.

55. The method of claim 42, wherein the at least one lipid comprises a phosphatidylcholine (PC), a phosphatidylglycerol (PG), cholesterol (Ch), a deuterated cholesterol, a diacylglycerol (DAG), a deuterated diacylglycerol, a phosphatidylserine (PS), a lysophosphatidylcholine (LPC), a ceramide (Cer), a phospatidylinositol (PI), a phosphatidic acid (PA), a phosphatidylethanolamine (PE), an acylcarnitine (AcCa), a lysophosphatidylethanolamine (LPE), a monoacylglycerol (MAG), a triacylglycerol (TAG), a dimethylammonium propane (DAP), a cholesteryl ester (ChE), a zymosterol (ZyE), a sterol ester (StE), a cardiolipin (CL), or a sphingomyelin (SM), or any derivative thereof.

56. The method of claim 42, wherein the at least one lipid comprises a ChE, a CER, a CL, a DAG, a LPC, a LPE, a PG, a PE, a PI, a SM, or a TAG.

57. The method of claim 42, wherein the performing mass spectrometry identifies at least about 1, 2, 3, 4, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, or more distinct lipids.

58. The method of claim 57, wherein the performing mass spectrometry comprises identifying at least about 10 cholesteryl esters (ChEs) in the biological sample.

59. The method of claim 57, wherein the performing mass spectrometry comprises identifying at least about 10 ceramides (CERs) in the biological sample.

60. The method of claim 57, wherein the performing mass spectrometry comprises identifying at least about 10 cardiolipins (CLs) in the biological sample.

61. The method of claim 57, wherein the performing mass spectrometry comprises identifying at least about 10 diacyl glycerols (DAGs) in the biological sample.
